(19) United States
(12) Patent Application Publication
(10) Pub. No.: US 2025/0388679 A1
(43) Pub. Date: Dec. 25, 2025

(54) SUBCUTANEOUS FCRN ANTAGONIST FORMULATIONS AND METHODS OF USE THEREOF

(71) Applicant: argenx BV, Ghent (BE)

(72) Inventor: Purve Patel, Ghent (BE)

(21) Appl. No.: 19/243,836

(22) Filed: Jun. 20, 2025

Related U.S. Application Data
(60) Provisional application No. 63/662,048, filed on Jun. 20, 2024.

(51) Int. Cl. C07K 16/28; A61K 9/00; A61K 38/47; A61K 39/00; A61K 47/02; A61K 47/18; A61K 47/22; A61P 21/04

(52) U.S. Cl. CPC C07K 16/283; A61K 9/0019; A61K 38/47; A61K 47/02; A61K 47/183; A61K 47/22; A61P 21/04; C12Y 302/01035; A61K 2039/505; A61K 2039/54; A61K 2039/545

(57) ABSTRACT

Provided herein are pharmaceutical compositions and approved products comprising an FcRn antagonist (e.g., efgartigimod, or a biosimilar version thereof) for subcutaneous administration and methods of use thereof for reducing the serum IgG level of a subject in need thereof (e.g., a subject having generalized myasthenia gravis (gMG)).

### CROSS REFERENCE TO RELATED APPLICATIONS

This application claims the benefit and priority to U.S. Provisional Patent Application No. 63/662,048, filed on Jun. 20, 2024, the contents of which is incorporated herein by reference in its entirety.

### SUBMISSION OF SEQUENCE LISTING XML

The content of the following submission of Sequence Listing XML is incorporated herein by reference in its entirety: a computer readable form (CRF) of the Sequence Listing (file name: 404373-T2407US_SL.xml, date created: May 30, 2025, size: 31,191 bytes).

### FIELD

The present disclosure relates to subcutaneous approved products comprising an FcRn antagonist (e.g., efgartigimod, or a biosimilar version thereof) and methods of use thereof for the treatment of generalized myasthenia gravis (gMG).

### BACKGROUND

There is a need in the art for approved products having subcutaneous formulations of FcRn antagonists such as efgartigimod for use in the treatment of gMG. An approved product is the final result of a multi-year effort requiring deep innovation and the generation of substantial amounts of data from studies and experiments testing an investigational agent to assess whether that investigational agent(s) may be approved to treat or mitigate a disease. This is true for Vyvgart Hytrulo® and its eventual FDA approval for treatment of gMG in June of 2023 was the result of years of effort, investment, and innovation. The data submitted to FDA proved that Vyvgart Hytrulo® was safe and effective for treating gMG under the conditions described in the product label (see Examples in this application).

The path to FDA approval is unpredictable and difficult. Most investigational agents fail during trials (more than 89%) and the approval process is a long and complex path. The odds that an investigational molecule will progress to becoming an approved product are quite low as shown in the report published by the Biotechnology Innovation Organization (go.bio.org/rs/490-EHZ-999/images/ClinicalDevelopmentSuccessRates2011_2020.pdf). For autoimmune diseases the likelihood of approval is 10.7% as detailed in the BIO study and FIG. 5a from that report (FIG. 1 herein).

### SUMMARY

The instant disclosure is directed to approved products comprising an FcRn antagonist (e.g., efgartigimod, or a biosimilar version thereof) and methods of use thereof for reducing the serum IgG level of a subject in need thereof (e.g., a subject having gMG).

Specifically, provided herein are approved products comprising: efgartigimod and a hyaluronidase, or a biosimilar version thereof, in solution for subcutaneous injection.

Also provided herein are biological products comprising efgartigimod and a hyaluronidase in a solution for subcutaneous injection, wherein the biological product is packaged in a single-dose vial. In some embodiments, the package includes a label that identifies the biological product as indicated for the treatment of gMG.

Kits comprising the disclosed approved products or biological products are also provided.

Provided herein are methods for treating gMG in a patient, the methods comprising administering the disclosed approved products to the patient in an amount and manner that is described in a label for the approved product.

Provided herein are methods of offering for sale an approved product, said methods comprising offering for sale such approved product, wherein an approved product label for a reference product for such approved product includes instructions for treating adult patients.

Also provided herein is an approved product comprising a pharmaceutical composition comprising about 180 mg/mL of a neonatal Fc receptor (FcRn) antagonist, about 2,000 U/mL hyaluronidase, about 1.4 mg/mL L-histidine, about 2.2 mg/mL L-histidine hydrochloride monohydrate, about 1.5 mg/mL L-methionine, about 0.4 mg/mL polysorbate 20, about 5.8 mg/mL sodium chloride, and about 20.5 mg/mL sucrose, at a pH of about 6.0, wherein the FcRn antagonist comprises or consists of a variant IgG Fc region, or FcRn-binding fragment thereof, wherein the variant IgG Fc region, or FcRn-binding fragment thereof, comprises or consists of a first Fc domain and a second Fc domain which form a homodimer or a heterodimer, and wherein the first Fc domain and the second Fc domain each comprise amino acids Y, T, E, K, F, and Y at EU positions 252, 254, 256, 433, 434, and 436, respectively.

In some embodiments, the first Fc domain and/or the second Fc domain comprise or consist of an amino acid sequence independently selected from the group consisting of SEQ ID NO: 1, SEQ ID NO: 2, SEQ ID NO: 3, and SEQ ID NO: 4. In some embodiments, the first Fc domain and the second Fc domain comprise or consist of an amino acid sequence independently selected from the group consisting of SEQ ID NO: 1, SEQ ID NO: 2, SEQ ID NO: 3, and SEQ ID NO: 4. In some embodiments, the first Fc domain and the second Fc domain comprise or consist of the amino acid sequence set forth in SEQ ID NO: 1. In some embodiments, the first Fc domain and the second Fc domain comprise or consist of the amino acid sequence set forth in SEQ ID NO: 2. In some embodiments, the first Fc domain and the second Fc domain comprise or consist of the amino acid sequence set forth in SEQ ID NO: 3. In some embodiments, the first Fc domain and the second Fc domain comprise or consist of the amino acid sequence set forth in SEQ ID NO: 4.

In some embodiments, the FcRn antagonist is a population of FcRn antagonist molecules, wherein each FcRn antagonist molecule in the population consists of a dimer of a first Fc domain and a second Fc domain, and wherein the population comprises a first subpopulation of FcRn antagonist molecules, wherein the amino acid sequences of both the first and the second Fc domains of the FcRn antagonist molecules in the first subpopulation consist of the amino acid sequence set forth in SEQ ID NO: 3; and at least one of: a second subpopulation of FcRn antagonist molecules, wherein the amino acid sequences of the first and the second Fc domains of the FcRn antagonist molecules in the second subpopulation consist of the amino acid sequences set forth in SEQ ID NO: 3 and SEQ ID NO: 12, respectively; a third subpopulation of FcRn antagonist molecules, wherein the amino acid sequences of the first and the second Fc domains of the FcRn antagonist molecules in the third subpopulation consist of the amino acid sequences set forth in SEQ ID NO: 3 and SEQ ID NO: 9, respectively; a fourth subpopulation of FcRn antagonist molecules, wherein the amino acid sequences of both the first and the second Fc domains of the FcRn antagonist molecules in the fourth subpopulation consist of the amino acid sequence set forth in SEQ ID NO: 3, and wherein two asparagine residues in each FcRn antagonist molecule in the fourth subpopulation are deaminated; a fifth subpopulation of FcRn antagonist molecules, wherein the amino acid sequences of the first and the second Fc domains of the FcRn antagonist molecules in the fifth subpopulation consist of the amino acid sequences set forth in SEQ ID NO: 3 and SEQ ID NO: 9, respectively, and wherein one asparagine residue in each FcRn antagonist molecule in the fifth subpopulation is deaminated; a sixth subpopulation of FcRn antagonist molecules, wherein the amino acid sequences of the first and the second Fc domains of the FcRn antagonist molecules in the sixth subpopulation consist of the amino acid sequences set forth in SEQ ID NO: 2 and SEQ ID NO: 3, respectively; a seventh subpopulation of FcRn antagonist molecules, wherein the amino acid sequences of the first and the second Fc domains of the FcRn antagonist molecules in the seventh subpopulation consist of the amino acid sequences set forth in SEQ ID NO: 2 and SEQ ID NO: 3, respectively, and wherein one methionine residue or one tryptophan residue in each FcRn antagonist molecule in the seventh subpopulation is oxidized; an eighth subpopulation of FcRn antagonist molecules, wherein the amino acid sequences of both the first and the second Fc domains of the FcRn antagonist molecules in the eighth subpopulation consist of the amino acid sequence set forth in SEQ ID NO: 2; a ninth subpopulation of FcRn antagonist molecules, wherein the amino acid sequences of the first and the second Fc domains of the FcRn antagonist molecules in the ninth subpopulation consist of the amino acid sequences set forth in SEQ ID NO: 2 and SEQ ID NO: 3, respectively, and wherein one methionine residue or one tryptophan residue in each FcRn antagonist molecule in the ninth subpopulation is oxidized; a tenth subpopulation of FcRn antagonist molecules, wherein the amino acid sequences of both the first and the second Fc domains of the FcRn antagonist molecules in the tenth subpopulation consist of the amino acid sequence set forth in SEQ ID NO: 3, and wherein two amino acid residues, independently selected from a methionine residue and a tryptophan residue, in each FcRn antagonist molecule in the tenth subpopulation is oxidized; and an eleventh subpopulation of FcRn antagonist molecules, wherein the amino acid sequences of the first and the second Fc domains of the FcRn antagonist molecules in the eleventh subpopulation consist of the amino acid sequences set forth in SEQ ID NO: 3 and SEQ ID NO: 6, respectively.

In some embodiments, the FcRn antagonist is efgartigimod, or a biosimilar version thereof. In some embodiments, the hyaluronidase is rHuPH20.

Also provided herein is an approved product comprising a pharmaceutical composition comprising 180 mg/mL of an FcRn antagonist, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, and 20.5 mg/mL sucrose, and water for injection, USP, at a pH of 6.0, wherein the FcRn antagonist is efgartigimod, or a biosimilar version thereof.

In some embodiments, the pharmaceutical composition is sterile, preservative free, yellowish, clear to opalescent.

In some embodiments, the pharmaceutical composition is for subcutaneous administration.

In some embodiments, the pharmaceutical composition is contraindicated in patients with serious hypersensitivity to efgartigimod products, to hyaluronidase, or to any composition excipients. In some embodiments, the hypersensitivity is anaphylaxis and/or hypotension leading to syncope.

Also provided herein is a single-dose vial comprising any approved product described herein. In some embodiments, the single-dose vial contains 5.6 mL of the approved product. In some embodiments, the single-dose vial contains 1008 mg of the FcRn antagonist and 11,200 U of the hyaluronidase.

Also provided herein is a method of reducing serum IgG levels in a subject in need thereof, the method comprising administering to the subject an effective amount of any approved product described herein, or an effective amount of an approved product contained within any single-dose vial described herein.

In some embodiments, the subject has generalized myasthenia gravis (gMG). In some embodiments, the subject is anti-acetylcholine receptor (AChR) antibody positive. In some embodiments, the subject is a human. In some embodiments, the subject is an adult human.

In some embodiments, the approved product is administered to the subject at a dose of 1,008 mg of the FcRn antagonist. In some embodiments, the approved product is administered to the subject once weekly.

Also provided herein is a method of treating gMG in a subject in need thereof, the method comprising administering to the subject an effective amount of any approved product described herein, or an effective amount of the approved product contained within any single-dose vial described herein.

In some embodiments, the subject is anti-AChR antibody positive. In some embodiments, the subject is a human. In some embodiments, the subject is an adult human.

In some embodiments, the approved product is administered to the subject at a dose of 1,008 mg of the FcRn antagonist. In some embodiments, the approved product is administered to the subject once weekly. In some embodiments, the approved product is administered to the subject once weekly for 4 weeks.

In some embodiments, the approved product is administered to the subject using a phased dosing schedule comprising a first treatment cycle and one or more subsequent treatment cycles, wherein the first treatment cycle and the one or more subsequent treatment cycles each independently comprise administration of 1-5 doses of the approved product within 1 month. In some embodiments, the first treatment cycle comprises weekly administration of the approved product for 4 weeks. In some embodiments, the one or more subsequent treatment cycles each comprise weekly administration of the approved product for 4 weeks. In some embodiments, each of the one or more subsequent treatment cycles is administered to the subject based on clinical evaluation. In some embodiments, each of the one or more subsequent treatment cycles is administered to the subject ≥50 days from the start of the previous treatment cycle.

Also provided herein is a method of treating gMG in a subject in need thereof, the method comprising administering an approved product comprising efgartigimod, or a biosimilar version thereof, to the subject at a dose of 1,008 mg via subcutaneous injection; monitoring the subject for a hypersensitivity reaction selected from the group consisting of anaphylaxis and hypotension leading to syncope; and initiating an appropriate measure to mitigate the hypersensitivity reaction when detected.

In some embodiments, the approved product comprises 180 mg/mL efgartigimod, or a biosimilar version thereof, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, 20.5 mg sucrose, and water for injection, USP, at a pH of 6.0.

In some embodiments, the approved product is administered to the subject via subcutaneous injection over 30 to 90 seconds.

In some embodiments, the approved product is administered to the subject once weekly. In some embodiments, the approved product is administered to the subject once weekly for 4 weeks.

In some embodiments, the approved product is administered to the subject using a phased dosing schedule comprising a first treatment cycle and one or more subsequent treatment cycles, wherein the first treatment cycle and the one or more subsequent treatment cycles each independently comprise administration of 1-5 doses of the approved product within 1 month. In some embodiments, the first treatment cycle comprises weekly administration of the approved product for 4 weeks. In some embodiments, the one or more subsequent treatment cycles each comprise weekly administration of the approved product for 4 weeks. In some embodiments, each of the one or more subsequent treatment cycles is administered to the subject based on clinical evaluation. In some embodiments, each of the one or more subsequent treatment cycles is administered to the subject ≥50 days from the start of the previous treatment cycle.

In some embodiments, the anaphylaxis and/or hypotension occurs within 1 hour of administration of the approved product to the subject.

In some embodiments, the subject is monitored for at least 30 minutes after administration of the approved product to the subject.

In some embodiments, the appropriate measure comprises permanent discontinuation of treatment with the approved product. In some embodiments, the appropriate measure comprises instituting appropriate measures or seeking medical attention.

In some embodiments, the subject is anti-AChR antibody positive. In some embodiments, the subject is a human. In some embodiments, the subject is an adult human.

Also provided herein is a method of treating gMG in a subject in need thereof, the method comprising administering an approved product comprising efgartigimod, or a biosimilar version thereof, to the subject at a dose of 1,008 mg via subcutaneous injection, wherein the subject has been informed of the signs and symptoms of a hypersensitivity reaction and advised to contact their healthcare provider immediately should they occur, wherein the hypersensitivity reaction is selected from the group consisting of anaphylaxis and hypotension leading to syncope.

In some embodiments, the approved product comprises 180 mg/mL efgartigimod, or a biosimilar version thereof, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, 20.5 mg sucrose, and water for injection, USP, at a pH of 6.0.

In some embodiments, the approved product is administered to the subject via subcutaneous injection over 30 to 90 seconds.

In some embodiments, the approved product is administered to the subject once weekly. In some embodiments, the approved product is administered to the subject once weekly for 4 weeks.

In some embodiments, the approved product is administered to the subject using a phased dosing schedule comprising a first treatment cycle and one or more subsequent treatment cycles, wherein the first treatment cycle and the one or more subsequent treatment cycles each independently comprise administration of 1-5 doses of the approved product within 1 month. In some embodiments, the first treatment cycle comprises weekly administration of the approved product for 4 weeks. In some embodiments, the one or more subsequent treatment cycles each comprise weekly administration of the approved product for 4 weeks. In some embodiments, each of the one or more subsequent treatment cycles is administered to the subject based on clinical evaluation. In some embodiments, each of the one or more subsequent treatment cycles is administered to the subject ≥50 days from the start of the previous treatment cycle.

In some embodiments, the subject is anti-AChR antibody positive. In some embodiments, the subject is a human. In some embodiments, the subject is an adult human.

Also provided herein is a method of treating gMG in a subject in need thereof, the method comprising administering an approved product comprising efgartigimod, or a biosimilar version thereof, to the subject at a dose of 1,008 mg via subcutaneous injection; monitoring the subject for an infusion-related reaction; and initiating an appropriate measure to mitigate the infusion-related reaction when detected.

In some embodiments, the method further comprises rechallenging the subject with the approved product when the infusion-related reaction is mild-to-moderate, wherein the rechallenging comprises one or more of the following: close clinical observation, slower infusion rate, and pre-medication. In some embodiments, the rechallenging comprises close clinical observation, slower infusion rate, and pre-medication. In some embodiments, the method further comprises initiating appropriate therapy when the infusion-related reaction is severe.

In some embodiments, the infusion-related reaction comprises one or more of the following: hypertension, chills, shivering, thoracic pain, abdominal pain, and back pain.

In some embodiments, the approved product comprises 180 mg/mL efgartigimod, or a biosimilar version thereof, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, 20.5 mg sucrose, and water for injection, USP, at a pH of 6.0.

In some embodiments, the approved product is administered to the subject via subcutaneous injection over 30 to 90 seconds.

In some embodiments, the approved product is administered to the subject once weekly. In some embodiments, the approved product is administered to the subject once weekly for 4 weeks.

In some embodiments, the approved product is administered to the subject using a phased dosing schedule comprising a first treatment cycle and one or more subsequent treatment cycles, wherein the first treatment cycle and the one or more subsequent treatment cycles each independently comprise administration of 1-5 doses of the approved product within 1 month. In some embodiments, the first treatment cycle comprises weekly administration of the approved product for 4 weeks. In some embodiments, the one or more subsequent treatment cycles each comprise weekly administration of the approved product for 4 weeks. In some embodiments, each of the one or more subsequent treatment cycles is administered to the subject based on clinical evaluation. In some embodiments, each of the one or more subsequent treatment cycles is administered to the subject ≥50 days from the start of the previous treatment cycle.

In some embodiments, the subject is anti-AChR antibody positive. In some embodiments, the subject is a human. In some embodiments, the subject is an adult human.

In some embodiments, the infusion-related reaction occurs within 1 hour of administration of the approved product to the subject.

In some embodiments, the subject is monitored for at least 30 minutes after administration of the approved product to the subject.

Also provided herein is a method of treating gMG in a subject in need thereof, the method comprising administering an approved product comprising efgartigimod, or a biosimilar version thereof, to the subject at a dose of 1,008 mg via subcutaneous injection, wherein the subject has been advised of the potential risk of an infusion-related reaction.

In some embodiments, the infusion-related reaction is selected from hypertension, chills, shivering, chest pain, abdominal pain, and back pain.

In some embodiments, the approved product comprises 180 mg/mL efgartigimod, or a biosimilar version thereof, 2,000 U rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, 20.5 mg sucrose, and water for injection, USP, at a pH of 6.0.

In some embodiments, the approved product is administered to the subject via subcutaneous injection over 30 to 90 seconds.

In some embodiments, the approved product is administered to the subject once weekly. In some embodiments, the approved product is administered to the subject once weekly for 4 weeks.

In some embodiments, the approved product is administered to the subject using a phased dosing schedule comprising a first treatment cycle and one or more subsequent treatment cycles, wherein the first treatment cycle and the one or more subsequent treatment cycles each independently comprise administration of 1-5 doses of the approved product within 1 month. In some embodiments, the first treatment cycle comprises weekly administration of the approved product for 4 weeks. In some embodiments, the one or more subsequent treatment cycles each comprise weekly administration of the approved product for 4 weeks. In some embodiments, each of the one or more subsequent treatment cycles is administered to the subject based on clinical evaluation. In some embodiments, each of the one or more subsequent treatment cycles is administered to the subject ≥50 days from the start of the previous treatment cycle.

In some embodiments, the subject is anti-AChR antibody positive. In some embodiments, the subject is a human. In some embodiments, the subject is an adult human.

Also provided herein is an approved product comprising efgartigimod and hyaluronidase, wherein the product is approved for treatment of gMG in adult patients who are anti-AChR antibody positive.

In some embodiments, the product is provided in a single-dose vial containing 180 mg/mL efgartigimod, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/ml sodium chloride, 20.5 mg sucrose, and water for injection, USP, at a pH of 6.0.

In some embodiments, the product is for administration via subcutaneous injection. In some embodiments, the product is administered via subcutaneous injection over 30 to 90 seconds.

In some embodiments, the product induces an MG-ADL response rate of 67.7% and a QMG response rate of 63.1% in a population of gMG patients who received 10 mg/kg efgartigimod, compared to a MG-ADL response rate of 29.7% and a QMG response rate of 14.1% in a population of gMG patients who received placebo.

In some embodiments, the product induces a hypersensitivity reaction selected from the group consisting of anaphylaxis and hypotension leading to syncope. In some embodiments, the anaphylaxis or hypotension leading to syncope occurs during or within 1 hour of administration of the product.

In some embodiments, the product is contraindicated in patients with serious hypersensitivity to efgartigimod alfa products or to any excipients in formulations thereof.

In some embodiments, the product induces an infusion-related reaction. In some embodiments, the infusion-related reaction comprises one or more of the following: hypertension, chills, shivering, thoracic pain, abdominal pain, and back pain. In some embodiments, one or more subsequent doses of the product are administered with close clinical observation, slower infusion rates, and pre-medications when a mild-to-moderate infusion-related reaction occurs during administration of the product.

Also provided is a biosimilar of any approved product described herein.

Also provided is a biological product that is bioequivalent to any approved product described herein.

Also provided is a kit comprising any approved product described herein, any single-dose vial described herein, any biosimilar described herein, or any biological product described herein, and a label.

In some embodiments, the label includes a contraindication in patients with serious hypersensitivity to efgartigimod alfa products, to hyaluronidase, or to any excipients in formulations thereof.

In some embodiments, the label includes a warning for hypersensitivity reactions selected from anaphylaxis and hypotension leading to syncope.

In some embodiments, the label includes a warning for infusion-related reactions selected from hypertension, chills, shivering, thoracic pain, abdominal pain, and back pain. In some embodiments, the label states to initiate appropriate therapy when a severe infusion-related reaction occurs. In some embodiments, the label states that patients may be rechallenged with close clinical observation, slower infusion rates, and pre-medications when a mild-to-moderate infusion-related reaction occurs.

In some embodiments, the label includes data demonstrating an MG-ADL response rate of 67.7% and a QMG response rate of 63.1% in a population of gMG patients who received 10 mg/kg efgartigimod, compared to a MG-ADL response rate of 29.7% and a QMG response rate of 14.1% in a population of gMG patients who received placebo.

Also provided is a method of treating gMG in a subject in need thereof, the method comprising administering any approved product described herein, any biosimilar described herein, or any biological product described herein to the subject.

In some embodiments, the approved product, biosimilar, or biological product is administered to the subject once weekly. In some embodiments, the approved product, biosimilar, or biological product is administered to the subject once weekly for 4 weeks.

In some embodiments, the approved product, biosimilar, or biological product is administered to the subject using a phased dosing schedule comprising a first treatment cycle and one or more subsequent treatment cycles, wherein the first treatment cycle and the one or more subsequent treatment cycles each independently comprise administration of 1-5 doses of the approved product, biosimilar, or biological product within 1 month. In some embodiments, the first treatment cycle comprises weekly administration of the approved product, biosimilar, or biological product for 4 weeks. In some embodiments, the one or more subsequent treatment cycles each comprise weekly administration of the approved product, biosimilar, or biological product for 4 weeks. In some embodiments, each of the one or more subsequent treatment cycles is administered to the subject based on clinical evaluation. In some embodiments, each of the one or more subsequent treatment cycles is administered to the subject ≥50 days from the start of the previous treatment cycle.

In some embodiments, the subject is anti-AChR antibody positive.

In some embodiments, the method further comprises monitoring the subject for a hypersensitivity reaction selected from the group consisting of anaphylaxis and hypotension leading to syncope; and initiating an appropriate measure to mitigate the hypersensitivity reaction when detected.

In some embodiments, the method further comprises monitoring the subject for an infusion-related reaction; and initiating an appropriate measure to mitigate the infusion-related reaction when detected. In some embodiments, the infusion-related reaction comprises one or more of the following: hypertension, chills, shivering, thoracic pain, abdominal pain, and back pain.

In some embodiments, the subject is a human. In some embodiments, the subject is an adult human.

### BRIEF DESCRIPTION OF DRAWINGS

FIG. 1 is a graph originally published as FIG. 5a in the report published by the Biotechnology Innovation Organization (go.bio.org/rs/490-EHZ-999/images/ClinicalDevelopmentSuccessRates2011_2020.pdf).

FIG. 2 is a graph showing the mean change in total MG-ADL from cycle 1 baseline over time in AChR-Ab positive gMG patients enrolled in the NCT03669588 study (modified intent-to-treat analysis set).

FIG. 3 is a set of graphs showing the percentage of patients with MG-ADL and QMG total score change 4 weeks post initial infusion of the first cycle in AChR-Ab positive gMG patients enrolled in the NCT03669588 study.

### DETAILED DESCRIPTION

### Definitions

Unless defined otherwise, all technical and scientific terms used herein have the same meaning as is commonly understood by one skilled in the art in the technical field of the invention.

As used herein, the term “approved product” refers to a biological product approved by the United States Food and Drug Administration (FDA) for introduction into commerce.

As used herein, the terms “biological product” or “biologic” can be used interchangeably to refer to a wide range of products such as vaccines, blood and blood components, allergenics, somatic cells, gene therapy, tissues, and recombinant therapeutic proteins. Biologics can be composed of sugars, proteins, or nucleic acids or complex combinations of these substances, or may be living entities such as cells and tissues. A biological product comprises a population of biological molecules (e.g., a population of individual protein molecules). Many therapeutic proteins are produced by recombinant DNA technology in animal or microbial host cells. During the manufacturing process, these engineered host cells make many copies of a therapeutic protein with the same amino acid sequence. Changes can occur to one or more amino acids in a given protein through post-translational modifications, such as glycosylation. Therefore, although a single protein is being manufactured, the individual protein molecules can be slightly different from each other. The resulting biological product ends up being a mix of these individual protein molecules with various sugars attached to them. Other post-translational modifications, such as oxidation or deamidation, or truncations may also be present. This is true for approved products, reference products, biosimilars, and interchangeable biosimilars.

A biological product may comprise other active or inactive ingredients. Inactive ingredients include formulation excipients. The term “dosage form,” as used herein refers to the physical form in which a biological product is produced and dispensed, such as, for example, an injectable, a tablet, or a capsule.

In some embodiments, the approved product is a biological product containing efgartigimod that has been approved for use in treating humans with myasthenia gravis and is formulated for delivery by subcutaneous injection in a 1,008 mg dose. An example of an “approved product” is the approved product described in Example 1.

In some embodiments, the approved product is provided in a single-dose vial containing 1008 mg of efgartigimod at a concentration of 180 mg/mL and 11,200 U of rHuPH20 at a concentration of 2,000/mL and each milliliter of solution comprises:
L-histidine (1.4 mg), L-histidine hydrochloride monohydrate (2.2 mg), L-methionine (1.5 mg), polysorbate 20 (0.4 mg), sodium chloride (5.8 mg), sucrose (20.5 mg), and water for injection, USP, at a pH of 6.0.

In some embodiments, the approved product is contraindicated in patients with serious hypersensitivity to efgartigimod alfa products, to hyaluronidase, or to any formulation excipients thereof. In some embodiments, hypersensitivity is anaphylaxis or hypotension leading to syncope.

In some embodiments, the approved product induces an infusion-related reaction. In some embodiments, the infusion-related reaction is hypertension, chills, shivering, thoracic pain, abdominal pain, or back pain.

In some embodiments, when the approved product is administered in a 1008 mg subcutaneous injection dose to subjects or a patient population having myasthenia gravis, it induces a statistically significant increase in MG-ADL response rate, and/or in QMG response rate compared to the respective response rate in a patient population receiving placebo. In some embodiments, when the approved product is administered in a 10 mg/kg infusion dose to subjects of a patient population having generalized myasthenia gravis, it induces an MG-ADL response rate of 67.7%, and a QMG response rate of 63.1%, compared to 29.7% and 14.1% response in a placebo population.

In some embodiments, when using the approved product, it is recommended to inform patients of hypersensitivity reactions and to monitor patients for hypersensitivity reactions during administration and for at least 30 minutes after administration. In some embodiments, when using the approved product, it is recommended to inform patients of hypersensitivity reactions and to monitor patients for hypersensitivity reactions during administration and for 1 hour after administration, where the hypersensitivity reactions are selected from the group consisting of anaphylaxis and hypotension leading to syncope. If hypersensitivity is observed in a patient during or after administration of the approved product, appropriate measures to mitigate said hypersensitivity reactions are instituted, which include discontinuing use.

In some embodiments, when using the approved product, it is recommended to monitor patients for infusion-related reactions during administration and for 1 hour after administration, where the reactions are selected from the group consisting of hypertension, chills, shivering, thoracic pain, abdominal pain, and back pain within an hour of administration. As used herein, the terms “thoracic pain” and “chest pain” are used interchangeably. If an infusion-related reaction is severe, the patient should discontinue treatment with the approved product and initiate appropriate therapy. If an infusion-related reaction is mild to moderate, the patient should be rechallenged with the approved product with close clinical observation, slower infusion rates, and pre-medications.

In some embodiments, the approved product is further defined as follows:
is provided in a single-dose vial containing 1008 mg of efgartigimod at a concentration of 180 mg/mL and each milliliter of solution comprises:
L-histidine (1.4 mg), L-histidine hydrochloride monohydrate (2.2 mg), L-methionine (1.5 mg), polysorbate 20 (0.4 mg), sodium chloride (5.8 mg), sucrose (20.5 mg), and water for injection, USP, at a pH of 6.0; and
is contraindicated in patients with serious hypersensitivity to efgartigimod alfa products, to hyaluronidase, or to any formulation excipients thereof; and
when administered in a 1008 mg subcutaneous injection to subjects or a patient population having myasthenia gravis, induces a statistically significant increase in MG-ADL response rate, and/or in QMG response rate compared to the respective response rate in a patient population receiving placebo; and
when administered, may induce a hypersensitivity reaction selected from anaphylaxis and hypotension leading to syncope during or within an hour of administration; and
when administered, may induce an infusion-related reaction selected from hypertension, chills, shivering, thoracic pain, abdominal pain, and back pain during or within an hour of administration; and
when using, it is recommended to inform patients of hypersensitivity reactions and to monitor patients for hypersensitivity reactions during administration and for at least 30 minutes after administration, where the hypersensitivity reactions are selected from the group consisting of anaphylaxis and hypotension leading to syncope, and if hypersensitivity is observed in a patient during or after administration of the approved product, appropriate measures to mitigate said hypersensitivity reactions are instituted, which include discontinuing use.

In some embodiments, the approved product is a product approved under Biologics License Application (BLA) 761304.

As used herein, the term “biosimilar” refers to a biological product approved by FDA under the Biologics Price Competition and Innovation Act that is highly similar to and has no clinically meaningful differences from an approved reference product. A biosimilar is a type of approved product. A biosimilar can be shown to be highly similar to the reference product by analyzing (i.e., characterizing) the structure and function of both the reference product and the proposed biosimilar and comparing characteristics of the products, such as purity, chemical identity, and bioactivity. Note that differences, such as oligosaccharide profiles, amino acid truncations or extensions, oxidation, deamidation, etc., may exist between biological products produced using different cell lines or manufacturing techniques and that a biological product may behave differently in patients if manufactured using a different cell line or a different methodology. However, differences, such as acceptable within-product variations, between the reference product and the proposed biosimilar can be present so long as the biosimilar is highly similar and has no clinically meaningful differences. Similarly, minor differences between the reference product and the proposed biosimilar in clinically inactive components are acceptable so long as the biosimilar is highly similar and has no clinically meaningful differences.

As used herein, “no clinically meaningful differences” is determined in terms of safety, purity, and potency. For example, a biosimilar is compared to and evaluated against a reference product to verify that the biosimilar has no clinically meaningful differences in terms of safety, purity, and potency from the reference product. In some embodiments, a determination of no clinically meaningful differences between a biosimilar and a reference product is based upon data derived from: (a) analytical studies that demonstrate that the biological product is highly similar to the reference product notwithstanding minor differences in clinically inactive components; (b) animal studies (including, for example, assessment of toxicity); and/or (c) a clinical study or studies (including, for example, assessment of immunogenicity and pharmacokinetics or pharmacodynamics) sufficient to demonstrate safety, purity, and potency in one or more appropriate conditions of use for which the reference product is licensed and for which licensure is sought for the biosimilar. A biosimilar may be an interchangeable biosimilar that may be substituted for the reference product at a pharmacy without intervention of a prescribing healthcare professional. To meet a standard of “interchangeability,” the biosimilar is expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. In some embodiments, the biosimilar utilizes the same mechanisms of action as the reference product for the proposed conditions of use, to the extent the mechanisms are known for the reference product. In some embodiments, the condition or conditions of use prescribed, recommended, or suggested in the labeling proposed for the biosimilar have been previously approved for the reference product. In some embodiments, the route of administration, the dosage form, and/or the strength of a biosimilar are the same as those of the reference product and the biosimilar is manufactured, processed, packed, or held in a facility that meets standards designed to assure that the biosimilar continues to be safe, pure, and potent.

As used herein, “bioequivalent” or “bioequivalence” is defined as the absence of a significant difference in the rate and extent to which the active ingredient or active moiety in pharmaceutical equivalents or pharmaceutical alternatives becomes available at the site(s) of drug action when administered at the same molar dose under similar conditions in an appropriately designed study.

A determination of “biosimilarity” or “bioequivalence” or “interchangeability” may be made according to any of the standards defined by the FDA. A company seeking approval to market a generic equivalent or biosimilar in the United States must refer to the reference product in its 351 (k) BLA. For example, a 351 (k) BLA applicant relies on the FDA's finding that a previously approved product, i.e., the reference product, is safe and effective, and must demonstrate, among other things, that the proposed biosimilar is the same as the reference product in certain ways. Specifically, with limited exceptions, a biological product for which an 351 (k) BLA is submitted must have, among other things, the same active ingredient(s), mechanism(s) of action for the proposed condition(s) of use (but only to the extent the mechanism(s) are known for the reference product), condition(s) of use, route of administration, dosage form, strength, and (with certain permissible differences) labeling as the reference product.

The term “reference product” refers to a product that is approved by a regulatory agency (e.g., the approved product) against which a proposed biosimilar is compared. A reference product is approved based on, among other things, a full complement of safety and effectiveness data. A proposed biosimilar product is compared to, and evaluated against, a reference product to ensure that the product is highly similar and has no clinically meaningful differences. In some embodiments, the reference product is an approved product as defined herein. In some embodiments, the reference product is a biological product, licensed in the United States under section 351 (a) of the Public Health Service (PHS) Act, against which a biological product is evaluated in a BLA submitted under section 351 (k) of the PHS Act (i.e., 351 (k) BLA).

As used herein, the terms “label,” “product label,” or “approved product label” refer to information provided to a patient and/or healthcare provider which provides relevant information regarding the approved product. Such information includes, without limitation, one or more of: the description of the approved product, clinical pharmacology, indications (uses for the approved product), contraindication (who should not take the approved product), warnings, precautions, adverse events (side effects), drug abuse and dependence, dosage and administration, use in pregnancy, use in nursing mothers, use in children and older patients, how the approved product is supplied, safety information for the patient, or any combination thereof. In some embodiments, the label identifies efgartigimod and provides instructions for its use in a patient.

As used herein, the term “FcRn” refers to a neonatal Fc receptor. Exemplary FcRn molecules include human FcRn encoded by the FCGRT gene as set forth in RefSeq NM 004107. The amino acid sequence of the corresponding protein is set forth in RefSeq NP_004098.

As used herein, the term “FcRn antagonist” refers to any agent that binds specifically to FcRn and inhibits the binding of immunoglobulin to FcRn (e.g., human FcRn). An example is efgartigimod.

As used herein, the term “Fc domain” refers to the portion of a single immunoglobulin heavy chain beginning in the hinge region and ending at the C-terminus of an antibody. Accordingly, a complete Fc domain comprises at least a portion of a hinge region (e.g., upper, middle, and/or lower hinge region), a CH2 domain, and a CH3 domain. In some embodiments, the term “Fc domain” refers to the portion of a single immunoglobulin heavy chain comprising both the CH2 and CH3 domains of an antibody. In some embodiments, the Fc domain comprises at least a portion of a hinge region (e.g., upper, middle, and/or lower hinge region), a CH2 domain, and a CH3 domain. In some embodiments, the Fc domain does not include the hinge region.

As used herein, the term “hinge region” refers to the portion of a heavy chain molecule that joins the CHI domain to the CH2 domain. In some embodiments, the hinge region is at most 70 amino acid residues in length. In some embodiments, this hinge region comprises approximately 11-17 amino acid residues and is flexible, thus allowing the two N-terminal antigen binding regions to move independently. In some embodiments, the hinge region is 12 amino acid residues in length. In some embodiments, the hinge region is 15 amino acid residues in length. In some embodiments, the hinge region is 62 amino acid residues in length. Hinge regions can be subdivided into three distinct domains: upper, middle, and lower hinge regions. The FcRn antagonists of the instant disclosure can include all or any portion of a hinge region. In some embodiments, the hinge region is from an IgG1 antibody. In some embodiments, the hinge region comprises the amino acid sequence of EPKSCDKTHTCPPCP (SEQ ID NO: 23).

As used herein, the term “Fc region” refers to the portion of an immunoglobulin formed by the Fc domains of its two heavy chains. The Fc region can be a wild-type Fc region (native Fc region) or a variant Fc region. A native Fc region is homodimeric. The Fc region can be derived from any native immunoglobulin. In some embodiments, the Fc region is formed from an IgG heavy chain constant region. In some embodiments, the Fc region is formed from an IgG heavy chain constant region. In some embodiments, the IgG heavy chain constant region is an IgG1, IgG2, IgG3, or IgG4 heavy chain constant region. In some embodiments, the Fc region is formed from an IgG1 heavy chain constant region. In some embodiments, the IgG1 heavy chain constant region comprises a Glml (a), Glm2 (x), Glm3 (f), or Glm17 (z) allotype, see, e.g., Jefferis and Lefranc, 2009 and de Taeye et al., 2020.

As used herein, the term “variant Fc region” refers to an Fc region with one or more alteration(s) relative to a native Fc region. Alterations can include amino acid substitutions, additions and/or deletions, linkage of additional moieties, and/or alteration of the native glycans. The term encompasses heterodimeric Fc regions where each of the constituent Fc domains is different. The term also encompasses single chain Fc regions where the constituent Fc domains are linked together by a linker moiety.

As used herein the term “FcRn binding fragment” refers to a portion of an Fc region that is sufficient to confer FcRn binding.

As used herein, the term “EU position” refers to the amino acid position in the EU numbering convention for the Fc region described in Edelman, G M et al., Proc. Natl. Acad. USA, 1969; 63, 78-85, and Kabat et al., in “Sequences of Proteins of Immunological Interest,” U.S. Dept. Health and Human Services, 5th edition, 1991.

As used herein, the term “baseline” refers to a measurement in a patient, e.g., in a patient's blood, prior to the first administration (e.g., intravenous administration) of a treatment (e.g., an FcRn antagonist).

As used herein, the term “treat,” “treating,” and “treatment” refer to therapeutic or preventative measures described herein. The methods of “treatment” employ administration of a polypeptide to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “dose” or “dosing” refers to an amount of an agent administered to a subject in a single administration.

As used herein, the terms “fixed dose” or “flat dose” both refer to a dose that does not vary based upon a characteristic (e.g., body mass, e.g., within a set range; sex; age, e.g., within a set range; etc.) of the subject.

As used herein, the term “subject” or “patient” or “participant” may be used interchangeably and includes any human or non-human animal. In an embodiment, the subject or patient or participant is a human or non-human mammal. In an embodiment, the subject or patient or participant is a human.

As used herein, the term “about” or “approximately” when referring to a measurable value, such as a dosage, encompasses variations of +5% of a given value or range, as are appropriate to perform the methods disclosed herein.

As used herein, the term “single-dose vial” refers to a vial of liquid medication intended for parenteral administration (e.g., subcutaneous injection) that is meant for use in a single patient for a single administration.

As used herein, the term “preservative free” refers to compositions (e.g., pharmaceutical compositions) that do not contain amounts of preservatives that are detectable using methods known in the art.

As used herein, the term “MG-ADL response” refers to a reduction of ≥2 points on MG-ADL score compared to baseline, for at least 4 consecutive weeks, where the first of the reductions occurs at the latest 1 week after the last treatment. The Myasthenia Gravis Activities of Daily Living (MG-ADL) scale is an 8-item patient-reported scale to assess MG symptoms and their effects on daily activities. It evaluates the capacity to perform different activities of daily living such as talking, chewing, swallowing, breathing, brushing the teeth/combing the hair, or arising from the chair and it also assesses double vision and eyelid droop. The 8 items of the MG-ADL were derived from symptom-based components of the original 13-item QMG to assess disability secondary to ocular (2 items), bulbar (3 items), respiratory (1 item), and gross motor or limb (2 items) impairment related to effects from MG. In this functional status instrument, each response is graded 0 (normal) to 3 (most severe). The range of total MG-ADL score is 0-24, where higher scores indicate more severe impairments.

As used herein, the term “QMG response” refers to a reduction of ≥3 points on QMG score compared to baseline, for at least 4 consecutive weeks, where the first of the reductions occurs at the latest 1 week after the last treatment. The Quantitative Myasthenia Gravis (QMG) scale is a standardized quantitative strength scoring system developed specifically for MG. The QMG has been validated and has been used by the investigators in several trials. It consists of 13 items that assess ocular, bulbar, and limb function. Out of the 13 items, 6 are timed tests of endurance measured in seconds. Each item has a possible score from 0-3. The total possible score is 39, where higher scores indicate more severe impairments.

As used herein, the term “Myasthenia Gravis Foundation of America (MGFA) clinical classification system” refers to the classification system designed by the Task Force of the Medical Scientific Advisory Board of the MGFA published in 2000 (Task Force of the Medical Scientific Advisory Board of the Myasthenia Gravis Foundation of America, Inc. Neurology 55:16-23 (2000)). The MGFA classification system defines classes of MG based on clinical symptoms as follows: Class I-any ocular muscle weakness, all other muscle strength is normal; Class II-mild weakness affecting other than ocular muscles, may also have ocular muscle weakness of any severity; Class IIa-predominantly affecting limb muscles, axial muscles, or both, may also have lesser involvement of oropharyngeal muscles, respiratory muscles, or both; Class IIb-predominantly affecting oropharyngeal muscles, respiratory muscles, or both, may also have lesser or equal involvement of limb muscles, axial muscles, or both; Class III-moderate weakness affecting other than ocular muscles, may also have ocular muscle weakness of any severity; Class IIIa-predominantly affecting limb muscles, axial muscles, or both, may also have lesser involvement of oropharyngeal muscles, respiratory muscles, or both; Class IIIb-predominantly affecting oropharyngeal muscles, respiratory muscles, or both, may also have lesser or equal involvement of limb muscles, axial muscles, or both; Class IV-severe weakness affecting other than ocular muscles, may also have ocular muscle weakness of any severity; Class IVa-predominantly affecting limb muscles, axial muscles, or both, may also have lesser involvement of oropharyngeal muscles, respiratory muscles, or both; Class IVb-predominantly affecting oropharyngeal muscles, respiratory muscles, or both, may also have lesser or equal involvement of limb muscles, axial muscles, or both; Class V-defined by intubation, with or without mechanical ventilation, except when employed during routine postoperative management.

### FcRn Antagonists

FcRn antagonists that are useful in the compositions, methods, and uses provided herein include variant Fc regions that bind to and inhibit FcRn. Any Fc region can be altered to produce a variant Fc region for use in the methods disclosed herein. In an embodiment, the Fc region is an IgG Fc region (e.g., a human IgG region). In an embodiment, the Fc region is selected from IgG1, IgG2, IgG3, and IgG4. In an embodiment, the Fc region is an IgG1 Fc region (e.g., a human IgG1 region). A variety of Fc region gene sequences (e.g., human constant region gene sequences) are available in the form of publicly accessible deposits.

An Fc region can be further truncated or internally deleted to produce a minimal FcRn binding fragment thereof. The ability of an Fc-region fragment to bind to FcRn can be determined using any art recognized binding assay (e.g., ELISA).

To enhance the manufacturability of the FcRn antagonists disclosed herein, it is preferable that the constituent Fc regions do not comprise any non-disulfide bonded cysteine residues. Accordingly, in an embodiment, the Fc regions do not comprise a free cysteine residue.

Any variant Fc region that binds specifically to FcRn with increased affinity and reduced pH dependence relative to the native (i.e., wild-type) Fc region can be used in the methods disclosed herein. In an embodiment, the variant Fc region comprises amino acid alterations, substitutions, insertions, and/or deletions that confer the desired characteristics. In some embodiments, the FcRn antagonist comprises or consists of a variant Fc region, or FcRn binding fragment thereof, which binds to FcRn with a higher affinity at pH 5.5 as compared to a corresponding wild-type Fc region. In some embodiments, the FcRn antagonist comprises or consists of a variant Fc region, or FcRn binding fragment thereof, which bind to FcRn with a higher affinity at pH 6.0 and/or at pH 7.4 as compared to a corresponding wild-type Fc region. In some embodiments, the FcRn antagonist comprises or consists of a variant Fc region, or FcRn binding fragment thereof, which bind to FcRn with a higher affinity at both acidic and neutral pH.

In some embodiments, the variant Fc region is derived from the Fc region of any native immunoglobulin. In some embodiments, the native immunoglobulin is a human immunoglobulin. In some embodiments, the immunoglobulin is IgG. In some embodiments, the immunoglobulin is human IgG. In some embodiments, the IgG is IgG1, IgG2, IgG3, or IgG4. In some embodiments, the human IgG is human IgG1, human IgG2, human IgG3, or human IgG4. In some embodiments, the variant Fc region varies from the human IgG1 Fc region. In some embodiments, the human IgG1 Fc region comprises a Glml (a), Glm2 (x), Glm3 (f), or Glm17 (z) allotype.

In an embodiment, the variant Fc region, or FcRn binding fragment thereof consists of two Fc domains.

In an embodiment, the variant Fc region comprises or consists of a first Fc domain and a second Fc domain which form a homodimer or heterodimer. In an embodiment, the first Fc domain and/or the second Fc domain comprise amino acids Y, T, E, K, and F at EU positions 252, 254, 256, 433, and 434, respectively. In an embodiment, the first Fc domain and/or the second Fc domain comprise amino acids Y, T, E, K, F, and Y at EU positions 252, 254, 256, 433, 434, and 436, respectively.

In some embodiments, the FcRn antagonists disclosed herein comprise or consist of at least one Fc domain, wherein the amino acid sequence of the at least one Fc domain comprises or consists of the amino acid sequence of SEQ ID NO: 22, provided below in Table 1.

TABLE 1

| | SEQ ID |
|---|---|
| Amino Acid Sequence | NO: |
| X₁X₂X₃X₄X₅X₆PPCPAPELLGGPSVFLFPPKPKDTLYITREPEVTCVVVDVSH | 22 |
| EDPEVKFNWYVDGVEVHNAKTKPREEQYNSTYRVVSVLTVLHQDWLNG | |
| KEYKCKVSNKALPAPIEKTISKAKGQPREPQVYTLPPSRDELTKNQVSLTC | |
| LVKGFYPSDIAVEWESNGQPENNYKTTPPVLDSDGSFFLYSKLTVDKSRW | |
| QQGNVFSCSVMHEALKFHYTQKSLSLSPX₇X₈ | |
| wherein: X₁ is D or absent; X₂ is K or absent; X₃ is T or | |
| absent; X₄ is H or absent; X₅ is T or absent; X₆ is C or | |
| absent; X₇ is G or absent; X₈ is K or absent. | |

In some embodiments, the FcRn antagonists disclosed herein comprise or consist of a variant Fc region comprising or consisting of a dimer of a first Fc domain and a second Fc domain, wherein the amino acid sequence of the first and second Fc domain comprises or consists of the amino acid sequence of SEQ ID NO: 22.

In some embodiments, the FcRn antagonists disclosed herein comprise or consist of an amino acid sequence at least 70%, 75%, 80%, 85%, 90%, 95%, 96%, 97%, 98%, 99%, or 100% identical to the amino acid sequence of a variant Fc region comprising or consisting of a dimer of a first Fc domain and a second Fc domain, wherein the amino acid sequence of the first and second Fc domain comprises or consists of an amino acid sequence independently selected from the group consisting of SEQ ID NOs: 1-21 (see Table 2 below). In some embodiments, the dimer is a heterodimer or a homodimer.

TABLE 2

| | SEQ |
|---|---|
| Amino Acid Sequence | ID NO: |
| CPPCPAPELLGGPSVFLFPPKPKDTLYITREPEVTCVVVDVSHEDPEVKFNW | 1 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSPG | |
| DKTHTCPPCPAPELLGGPSVFLFPPKPKDTLYITREPEVTCVVVDVSHEDPE | 2 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPGK | |
| DKTHTCPPCPAPELLGGPSVFLFPPKPKDTLYITREPEVTCVVVDVSHEDPE | 3 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPG | |
| CPPCPAPELLGGPSVFLFPPKPKDTLYITREPEVTCVVVDVSHEDPEVKFNW | 4 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSPGK | |
| DKTHTCPPCPAPELLGGPSVFLFPPKPKDTLYITREPEVTCVVVDVSHEDPE | 5 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSP | |
| KTHTCPPCPAPELLGGPSVFLFPPKPKDTLYITREPEVTCVVVDVSHEDPEV | 6 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPGK | |
| KTHTCPPCPAPELLGGPSVFLFPPKPKDTLYITREPEVTCVVVDVSHEDPEV | 7 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPG | |
| KTHTCPPCPAPELLGGPSVFLFPPKPKDTLYITREPEVTCVVVDVSHEDPEV | 8 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSP | |
| THTCPPCPAPELLGGPSVFLFPPKPKDTLYITREPEVTCVVVDVSHEDPEVKF | 9 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSPGK | |
| THTCPPCPAPELLGGPSVFLFPPKPKDTLYITREPEVTCVVVDVSHEDPEVKF | 10 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSPG | |
| THTCPPCPAPELLGGPSVFLFPPKPKDTLYITREPEVTCVVVDVSHEDPEVKF | 11 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSP | |
| TCPPCPAPELLGGPSVFLFPPKPKDTLYITREPEVTCVVVDVSHEDPEVKFN | 12 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPGK | |
| TCPPCPAPELLGGPSVFLFPPKPKDTLYITREPEVTCVVVDVSHEDPEVKFN | 13 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPG | |
| TCPPCPAPELLGGPSVFLFPPKPKDTLYITREPEVTCVVVDVSHEDPEVKFN | 14 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSP | |
| PPCPAPELLGGPSVFLFPPKPKDTLYITREPEVTCVVVDVSHEDPEVKFNWY | 15 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSPGK | |
| PPCPAPELLGGPSVFLFPPKPKDTLYITREPEVTCVVVDVSHEDPEVKFNWY | 16 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSPG | |
| PPCPAPELLGGPSVFLFPPKPKDTLYITREPEVTCVVVDVSHEDPEVKFNWY | 17 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSP | |
| HTCPPCPAPELLGGPSVFLFPPKPKDTLYITREPEVTCVVVDVSHEDPEVKFN | 18 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPGK | |
| HTCPPCPAPELLGGPSVFLFPPKPKDTLYITREPEVTCVVVDVSHEDPEVKFN | 19 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPG | |
| HTCPPCPAPELLGGPSVFLFPPKPKDTLYITREPEVTCVVVDVSHEDPEVKFN | 20 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSP | |
| CPPCPAPELLGGPSVFLFPPKPKDTLYITREPEVTCVVVDVSHEDPEVKFNW | 21 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSP | |

In an embodiment, the first Fc domain and/or the second Fc domain comprise an amino acid sequence independently selected from the group consisting of SEQ ID NOs: 1, 2, 3, and 4. In an embodiment, the first Fc domain and the second Fc domain comprise an amino acid sequence independently selected from the group consisting of SEQ ID NOs: 1, 2, 3, and 4.

In some embodiments, the FcRn antagonist comprises a population of FcRn antagonist molecules. In some embodiments, an FcRn antagonist comprising a first Fc domain and a second Fc domain comprising an amino acid sequence independently selected from the group consisting of SEQ ID NOs: 1, 2, 3, and 4 is the predominant FcRn antagonist molecule in the population of FcRn antagonist molecules. In some embodiments, the predominant FcRn antagonist molecule makes up at least 50%, 55%, 60%, 65%, 70%, 75%, 80%, 85%, 90%, 95%, 96%, 97%, 98%, or 99% of the population of FcRn antagonist molecules.

In an embodiment, the amino acid sequence of the Fc domains of the variant Fc region comprises the amino acid sequence of SEQ ID NO: 1. In an embodiment, the amino acid sequence of the Fc domains of the variant Fc region consists of the amino acid sequence of SEQ ID NO: 1.

In an embodiment, the amino acid sequence of the Fc domains of the variant Fc region comprises the amino acid sequence of SEQ ID NO: 2. In an embodiment, the amino acid sequence of the Fc domains of the variant Fc region consists of the amino acid sequence of SEQ ID NO: 2.

In an embodiment, the amino acid sequence of the Fc domains of the variant Fc region comprises the amino acid sequence of SEQ ID NO: 3. In an embodiment, the amino acid sequence of the Fc domains of the variant Fc region consists of the amino acid sequence of SEQ ID NO: 3.

In an embodiment, the amino acid sequence of the Fc domains of the variant Fc region comprises the amino acid sequence of SEQ ID NO: 4. In an embodiment, the amino acid sequence of the Fc domains of the variant Fc region consists of the amino acid sequence of SEQ ID NO: 4.

In certain embodiments, the variant Fc region is a heterodimer, where the constituent Fc domains are different from each other. Methods of producing Fc heterodimers are known in the art (see, e.g., U.S. Pat. No. 8,216,805, which is incorporated by reference herein in its entirety). In an embodiment, the FcRn antagonist consists of a variant Fc region, wherein the variant Fc region consists of two Fc domains which form a heterodimer, wherein the amino acid sequence of each of the Fc domains is independently selected from SEQ ID NO: 1, SEQ ID NO: 2, SEQ ID NO: 3, or SEQ ID NO: 4. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 1, and the amino acid sequence of the second Fc domain consists of or comprises the amino acid sequence of SEQ ID NO: 2, SEQ ID NO: 3, or SEQ ID NO: 4. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 2, and the amino acid sequence of the second Fc domain consists of or comprises the amino acid sequence of SEQ ID NO: 1, SEQ ID NO: 3, or SEQ ID NO: 4. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 3, and the amino acid sequence of the second Fc domain consists of or comprises the amino acid sequence of SEQ ID NO: 1, SEQ ID NO: 2, or SEQ ID NO: 4. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 4, and the amino acid sequence of the second Fc domain consists of or comprises the amino acid sequence of SEQ ID NO: 1, SEQ ID NO: 2, or SEQ ID NO: 3.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 1.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 2.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 3.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 4.

In some embodiments, the FcRn antagonist comprises glycanation on one or both of the Fc domains. In some embodiments, the FcRn antagonist molecules comprise glycanation at EU position 297 on one or both of the Fc domains. In some embodiments, the glycanation comprises an N-glycan. In some embodiments, the N-glycan comprises a G0F N-glycan, G1F N-glycan, G2F N-glycan, or G0 N-glycan.

In some embodiments, the FcRn antagonist comprises or consists of a population of FcRn antagonists, wherein at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, or at least 57% of the population of Fc domains of the FcRn antagonists comprise galactose. In some embodiments, the population comprises or consists of FcRn antagonists, wherein at least 88%, at least 89%, at least 90%, at least 91%, at least 92%, at least 93%, at least 94%, at least 95%, at least 96%, at least 97%, at least 98%, or at least 99% of the population of Fc domains of the FcRn antagonists comprise fucose.

In some embodiments, the FcRn antagonist lacks an amino acid at EU position 441 of one or both Fc domains. In some embodiments, the FcRn antagonist comprises glycine and lysine at EU positions 440 and 441, respectively. In some embodiments, the FcRn antagonist lacks amino acids at EU positions 440 and 441. In some embodiments, the FcRn antagonist comprises amidated proline at EU position 439. In some embodiments, the FcRn antagonist lacks amino acids at EU positions 440 and 441 and comprises amidated proline at EU position 439.

In some embodiments, the FcRn antagonist comprises aspartate, lysine, threonine, histidine, threonine, and cysteine at EU positions 221, 222, 223, 224, 225, and 226, respectively. In some embodiments, the FcRn antagonist lacks an amino acid at EU positions 221, and comprises lysine, threonine, histidine, threonine, and cysteine at EU positions 222, 223, 224, 225, and 226, respectively. In some embodiments, the FcRn antagonist lacks amino acids at EU positions 221 and 222, and comprises threonine, histidine, threonine, and cysteine at EU positions 223, 224, 225, and 226, respectively. In some embodiments, the FcRn antagonist lacks amino acids at EU positions 221-224, and comprises threonine and cysteine at EU positions 225 and 226, respectively. In some embodiments, the FcRn antagonist lacks amino acids at EU positions 221, 222, 223, 224, 225, and 226.

In some embodiments, the FcRn antagonist is a population of FcRn antagonist molecules. In some embodiments, the population of FcRn antagonist molecules comprises or consists of multiple subpopulations of FcRn antagonist molecules. In some embodiments, the population of FcRn antagonist molecules comprises or consists of 2, 3, 4, 5, 6, 7, 8, 9, 10, 11, 12, 13, 14, 15, 16, 17, 18, 19, or 20 subpopulations. In some embodiments, the population of FcRn antagonist molecules comprises or consists of 2, 3, 4, 5, 6, 7, 8, 9, 10, or 11 subpopulations.

In some embodiments, a first subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3.

In some embodiments, a second subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 13, respectively.

In some embodiments, a third subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 10, respectively.

In some embodiments, a fourth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein two asparagine residues in each FcRn antagonist molecule in the fourth subpopulation are deaminated.

In some embodiments, a fifth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein one asparagine residue in each FcRn antagonist molecule in the fifth subpopulation are deaminated.

In some embodiments, a sixth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein one methionine residue or one tryptophan residue in each FcRn antagonist molecule in the seventh subpopulation is oxidized.

In some embodiments, an eighth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 2.

In some embodiments, a ninth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 7, respectively.

In some embodiments, a tenth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively, and wherein one methionine residue or one tryptophan residue in each FcRn antagonist molecule in the tenth subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein two amino acid residues, independently selected from a methionine residue or a tryptophan residue, in each FcRn antagonist molecule in the eleventh subpopulation is oxidized.

In some embodiments, a first subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3.

In some embodiments, a second subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 13, respectively.

In some embodiments, a third subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 10, respectively.

In some embodiments, a fourth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3, and wherein two asparagine residues in each FcRn antagonist molecule in the fourth subpopulation are deaminated.

In some embodiments, a fifth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NO: 3, and wherein one asparagine residue in each FcRn antagonist molecule in the fifth subpopulation is deaminated.

In some embodiments, a sixth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NO: 3, and wherein one methionine residue or one tryptophan residue in each FcRn antagonist molecule in the seventh subpopulation is oxidized.

In some embodiments, an eighth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 2.

In some embodiments, a ninth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 7, respectively.

In some embodiments, a tenth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively, and wherein one methionine residue or one tryptophan residue in each FcRn antagonist molecule in the tenth subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3, and wherein two amino acid residues, independently selected from a methionine residue or a tryptophan residue in each FcRn antagonist molecule in the eleventh subpopulation is oxidized.

In some embodiments, the population of FcRn antagonist molecules comprises or consists of the first subpopulation combined with one of the second, third, fourth, fifth, sixth, seventh, eighth, ninth, tenth, or eleventh subpopulations. In some embodiments, the population of FcRn antagonist molecules comprises or consists of the first subpopulation combined with two of the second, third, fourth, fifth, sixth, seventh, eighth, ninth, tenth, or eleventh subpopulations. In some embodiments, the population of FcRn antagonist molecules comprises or consists of the first subpopulation combined with three of the second, third, fourth, fifth, sixth, seventh, eighth, ninth, tenth, or eleventh subpopulations. In some embodiments, the population of FcRn antagonist molecules comprises or consists of the first subpopulation combined with four of the second, third, fourth, fifth, sixth, seventh, eighth, ninth, tenth, or eleventh subpopulations. In some embodiments, the population of FcRn antagonist molecules comprises or consists of the first subpopulation combined with five of the second, third, fourth, fifth, sixth, seventh, eighth, ninth, tenth, or eleventh subpopulations. In some embodiments, the population of FcRn antagonist molecules comprises or consists of the first subpopulation combined with six of the second, third, fourth, fifth, sixth, seventh, eighth, ninth, tenth, or eleventh subpopulations. In some embodiments, the population of FcRn antagonist molecules comprises or consists of the first subpopulation combined with seven of the second, third, fourth, fifth, sixth, seventh, eighth, ninth, tenth, or eleventh subpopulations. In some embodiments, the population of FcRn antagonist molecules comprises or consists of the first subpopulation combined with eight of the second, third, fourth, fifth, sixth, seventh, eighth, ninth, tenth, or eleventh subpopulations. In some embodiments, the population of FcRn antagonist molecules comprises or consists of the first subpopulation combined with nine of the second, third, fourth, fifth, sixth, seventh, eighth, ninth, tenth, or eleventh subpopulations. In some embodiments, the population of FcRn antagonist molecules comprises or consists of the first subpopulation combined with all of the second, third, fourth, fifth, sixth, seventh, eighth, ninth, tenth, or eleventh subpopulations.

In some embodiments, the population comprises or consists of the first and second subpopulations. In some embodiments, the population comprises or consists of the first and third subpopulations. In some embodiments, the population comprises or consists of the first and fourth subpopulations. In some embodiments, the population comprises or consists of the first and fifth subpopulations. In some embodiments, the population comprises or consists of the first and sixth subpopulations. In some embodiments, the population comprises or consists of the first and seventh subpopulations. In some embodiments, the population comprises or consists of the first and eighth subpopulations. In some embodiments, the population comprises or consists of the first and ninth subpopulations. In some embodiments, the population comprises or consists of the first and tenth subpopulations. In some embodiments, the population comprises or consists of the first and eleventh subpopulations. In some embodiments, the populations listed above further comprise or consist of 1, 2, 3, 4, 5, 6, 7, 8, or 9 additional subpopulations. In some embodiments, these additional subpopulations are one or more of those described above.

In some embodiments, the population comprises or consists of the first and seventh, ninth, or eleventh subpopulations. In some embodiments, the population comprises or consists of the first, seventh, ninth, and eleventh subpopulations.

In some embodiments, the first subpopulation makes up at least 40%, at least 45%, at least 50%, at least 55%, at least 60%, at least 65%, at least 70%, at least 75%, at least 80%, at least 85%, or at least 90% of the population of FcRn antagonist molecules. In some embodiments, the first subpopulation makes up about 40%, about 45%, about 50%, about 55%, about 60%, about 65%, about 70%, about 75%, about 80%, about 85%, or about 90% of the population of FcRn antagonist molecules. In some embodiments, the first subpopulation makes up 40%, 45%, 50%, 55%, 60%, 65%, 70%, 75%, 80%, 85%, or 90% of the population of FcRn antagonist molecules. In some embodiments, the first subpopulation makes up 40%-90%, 50%-80%, or 55%-70% of the population of FcRn antagonist molecules. In some embodiments, the first subpopulation makes up 56.9%-68.3% or 59.5%-67.9% of the population of FcRn antagonist molecules.

In some embodiments, the second subpopulation makes up less than 3.0%, less than 2.5%, less than 2.0%, less than 1.5%, less than 1%, or less than 0.5% of the population of FcRn antagonist molecules. In some embodiments, the second subpopulation makes up about 3.0%, about 2.5%, about 2.0%, about 1.5%, about 1%, or about 0.5% of the population of FcRn antagonist molecules. In some embodiments, the second subpopulation makes up 3.0%, 2.5%, 2.0%, 1.5%, 1%, or 0.5% of the population of FcRn antagonist molecules. In some embodiments, the second subpopulation makes up 0.5%-3.0%, 1.0%-2.5%, or 1.0%-2.0% of the population of FcRn antagonist molecules. In some embodiments, the second subpopulation makes up 0.8%-2.0% or 0.8%-2.1% of the population of FcRn antagonist molecules.

In some embodiments, the third subpopulation makes up less than 3.0%, less than 2.5%, less than 2.0%, less than 1.5%, less than 1%, or less than 0.5% of the population of FcRn antagonist molecules. In some embodiments, the third subpopulation makes up about 3.0%, about 2.5%, about 2.0%, about 1.5%, about 1%, or about 0.5% of the population of FcRn antagonist molecules. In some embodiments, the third subpopulation makes up 3.0%, 2.5%, 2.0%, 1.5%, 1%, or 0.5% of the population of FcRn antagonist molecules. In some embodiments, the third subpopulation makes up 0.5%-3.0%, 1.0%-2.5%, or 1.0%-2.0% of the population of FcRn antagonist molecules. In some embodiments, the third subpopulation makes up 1.1%-2.1% or 1.0%-1.9% of the population of FcRn antagonist molecules.

In some embodiments, the fourth subpopulation makes up less than 5%, less than 4%, less than 3%, less than 2%, or less than 1% of the population of FcRn antagonist molecules. In some embodiments, the fourth subpopulation makes up about 5%, about 4%, about 3%, about 2%, or about 1% of the population of FcRn antagonist molecules. In some embodiments, the fourth subpopulation makes up 5%, 4%, 3%, 2%, or 1% of the population of FcRn antagonist molecules. In some embodiments, the fourth subpopulation makes up 1%-5%, 2%-4%, or 2%-3% of the population of FcRn antagonist molecules. In some embodiments, the fourth subpopulation makes up 2.1%-3.2% or 2.0%-3.1% of the population of FcRn antagonist molecules.

In some embodiments, the fifth subpopulation makes up less than 12%, less than 11%, less than 10%, less than 9%, less than 8%, less than 7%, less than 6%, or less than 5% of the population of FcRn antagonist molecules. In some embodiments, the fifth subpopulation makes up about 12%, about 11%, about 10%, about 9%, about 8%, about 7%, about 6%, or about 5% of the population of FcRn antagonist molecules. In some embodiments, the fifth subpopulation makes up 12%, 11%, 10%, 9%, 8%, 7%, 6%, or 5% of the population of FcRn antagonist molecules. In some embodiments, the fifth subpopulation makes up 5%-12%, 6%-10%, or 7%-8% of the population of FcRn antagonist molecules. In some embodiments, the fifth subpopulation makes up 6.8%-9.4% or 6.9%-8.7% of the population of FcRn antagonist molecules.

In some embodiments, the sixth subpopulation makes up less than 17%, less than 16%, less than 15%, less than 14%, less than 13%, less than 12%, less than 11%, less than 10%, less than 9%, less than 8%, less than 7%, or less than 6% of the population of FcRn antagonist molecules. In some embodiments, the sixth subpopulation makes up about 17%, about 16%, about 15%, about 14%, about 13%, about 12%, about 11%, about 10%, about 9%, about 8%, about 7%, or about 6% of the population of FcRn antagonist molecules. In some embodiments, the sixth subpopulation makes up 17%, 16%, 15%, 14%, 13%, 12%, 11%, 10%, 9%, 8%, 7%, or 6% of the population of FcRn antagonist molecules. In some embodiments, the sixth subpopulation makes up 7%-17%, 10%-15%, or 11%-12% of the population of FcRn antagonist molecules. In some embodiments, the sixth subpopulation makes up 7.0%-14.0% or 10.0%-14.4% of the population of FcRn antagonist molecules.

In some embodiments, the seventh subpopulation makes up less than 6.0%, less than 5.5%, less than 5.0%, less than 4.5%, less than 4.0%, less than 3.5%, less than 3.0%, less than 2.5%, less than 2.0%, less than 1.5%, less than 1%, or less than 0.5% of the population of FcRn antagonist molecules. In some embodiments, the seventh subpopulation makes up about 6.0%, about 5.5%, about 5.0%, about 4.5%, about 4.0%, about 3.5%, about 3.0%, about 2.5%, about 2.0%, about 1.5%, about 1%, or about 0.5% of the population of FcRn antagonist molecules. In some embodiments, the seventh subpopulation makes up 6.0%, 5.5%, 5.0%, 4.5%, 4.0%, 3.5%, 3.0%, 2.5%, 2.0%, 1.5%, 1%, or 0.5% of the population of FcRn antagonist molecules. In some embodiments, the seventh subpopulation makes up 0.5%-5.5%, 1.0%-3.0%, or 1.5%-2.5% of the population of FcRn antagonist molecules. In some embodiments, the seventh subpopulation makes up 1.5%-5.5% or 1.4%-4.9% of the population of FcRn antagonist molecules.

In some embodiments, the eighth subpopulation makes up less than 7.5%, less than 7.0%, less than 6.5%, less than 6.0%, less than 5.5%, less than 5.0%, less than 4.5%, less than 4.0%, less than 3.5%, less than 3.0%, or less than 2.5% of the population of FcRn antagonist molecules. In some embodiments, the eighth subpopulation makes up about 7.5%, about 7.0%, about 6.5%, about 6.0%, about 5.5%, about 5.0%, about 4.5%, about 4.0%, about 3.5%, about 3.0%, or about 2.5% of the population of FcRn antagonist molecules. In some embodiments, the eighth subpopulation makes up 7.5%, 7.0%, 6.5%, 6.0%, 5.5%, 5.0%, 4.5%, 4.0%, 3.5%, 3.0%, or 2.5% of the population of FcRn antagonist molecules. In some embodiments, the eighth subpopulation makes up 2.5%-7.5%, 3.0%-5.0%, or 3.5%-4.5% of the population of FcRn antagonist molecules. In some embodiments, the eighth subpopulation makes up 2.9%-7.4% or 3.0%-6.3% of the population of FcRn antagonist molecules.

In some embodiments, the ninth subpopulation makes up less than 3.5%, less than 3.0%, less than 2.5%, less than 2.0%, less than 1.5%, less than 1%, or less than 0.5% of the population of FcRn antagonist molecules. In some embodiments, the ninth subpopulation makes up about 3.5%, about 3.0%, about 2.5%, about 2.0%, about 1.5%, about 1%, or about 0.5% of the population of FcRn antagonist molecules. In some embodiments, the ninth subpopulation makes up 3.5%, 3.0%, 2.5%, 2.0%, 1.5%, 1%, or 0.5% of the population of FcRn antagonist molecules. In some embodiments, the ninth subpopulation makes up 0.5%-3.5%, 1.5%-2.0%, or 1.0%-1.5% of the population of FcRn antagonist molecules. In some embodiments, the ninth subpopulation makes up 0.4%-3.2% or 0.5%-2.6% of the population of FcRn antagonist molecules.

In some embodiments, the tenth subpopulation makes up less than 2.0%, less than 1.5%, less than 1%, or less than 0.5% of the population of FcRn antagonist molecules. In some embodiments, the tenth subpopulation makes up about 2.0%, about 1.5%, about 1%, or about 0.5% of the population of FcRn antagonist molecules. In some embodiments, the tenth subpopulation makes up 2.0%, 1.5%, 1%, or 0.5% of the population of FcRn antagonist molecules. In some embodiments, the tenth subpopulation makes up 0.5%-2.0%, 0.5%-1.5%, or 1.0%-1.5% of the population of FcRn antagonist molecules.

In some embodiments, the eleventh subpopulation makes up less than 2.0%, less than 1.5%, less than 1%, or less than 0.5% of the population of FcRn antagonist molecules. In some embodiments, the eleventh subpopulation makes up about 2.0%, about 1.5%, about 1%, or about 0.5% of the population of FcRn antagonist molecules. In some embodiments, the eleventh subpopulation makes up 2.0%, 1.5%, 1%, or 0.5% of the population of FcRn antagonist molecules. In some embodiments, the eleventh subpopulation makes up 0.5%-2.0%, 0.5%-1.5%, or 1.0%-1.5% of the population of FcRn antagonist molecules.

In some embodiments, the population of FcRn antagonist molecules comprises one or more of the FcRn antagonists described herein. In some embodiments, the FcRn antagonist is any of those described in International Patent Application No. PCT/IB2023/000696, filed on Nov. 14, 2023, incorporated herein by reference in its entirety. In some embodiments, the FcRn antagonist is a population of FcRn antagonists as described in International Patent Application No. PCT/IB2023/000696, filed on Nov. 14, 2023, incorporated herein by reference in its entirety.

In an embodiment, the FcRn antagonist is efgartigimod (CAS Registry No. 1821402-21-4). Efgartigimod is described in further detail herein below. The term “efgartigimod” as used herein is interchangeable with “efgartigimod alfa”. In some embodiments, efgartigimod is efgartigimod alfa-fcab.

### Pharmaceutical Compositions

The instant disclosure provides pharmaceutical compositions comprising an FcRn antagonist.

In an embodiment, the FcRn antagonist is efgartigimod, or a biosimilar version thereof. Efgartigimod (ARGX-113) is a modified human immunoglobulin (Ig) gamma (IgG) 1-derived Fc of the za allotype that binds with nanomolar affinity to human FcRn. Efgartigimod encompasses the IgG1 Fc region (encompassing residues of SEQ ID NO: 2) and has been engineered using ABDEG™ technology to increase its affinity for FcRn at both physiological and acidic pH, see Vaccaro C et al., Nat Biotechnol. 2005; 23 (10): 1283. See also U.S. Pat. No. 10,316,073, the contents of which are incorporated by reference herein in their entirety. The increased affinity for FcRn of efgartigimod at both acidic and physiological pH results in a blockage of FcRn-mediated recycling of IgGs.

Efgartigimod alfa is a human recombinant immunoglobulin G1 (IgG1)-derived Fc fragment produced in Chinese hamster ovary (CHO) cells by recombinant DNA technology. Efgartigimod alfa is a human IgG1 antibody fragment engineered for increased affinity to the neonatal Fc Receptor (FcRn).

Efgartigimod alfa-fcab is a human immunoglobulin G1 (IgG1)-derived Fc fragment (fragment, crystallized) of the za allotype. The efgartigimod alfa-fcab Fc fragment is a homodimer consisting of two identical peptide chains each consisting of 227 amino acids linked together by two interchain disulfide bonds with affinity for FcRn. The molecule weight of efgartigimod alfa-fcab is approximately 54 kDa.

Efgartigimod is a prescription medicine registered as VYVGART®, which is approved in the United States, Europe, United Kingdom, China, Canada, and Israel for the treatment of adults with gMG who are anti-AChR antibody positive, and in Japan for the treatment of adults with gMG who do not have sufficient response to steroids or non-steroidal immunosuppressive therapies (ISTs). Efgartigimod is also approved in Japan for the treatment of chronic ITP. Efgartigimod is under development for both the intravenous (IV) and subcutaneous (SC) administration route in multiple indications.

In an embodiment, the pharmaceutical composition is a biological product that is highly similar to and has no clinically meaningful differences from efgartigimod.

In some embodiments, the pharmaceutical composition comprises an FcRn antagonist in an amount from about 20 mg to about 4,000 mg. In some embodiments, the pharmaceutical composition comprises an FcRn antagonist in an amount from about 300 mg to about 2,000 mg. In some embodiments, the pharmaceutical composition comprises an FcRn antagonist in an amount from about 500 mg to about 1500 mg.

In some embodiments, the pharmaceutical composition comprises about 1,000 mg of an FcRn antagonist. In some embodiments, the pharmaceutical composition comprises 1,000 mg of an FcRn antagonist. In some embodiments, the FcRn antagonist is efgartigimod, or a biosimilar version thereof.

In some embodiments, the pharmaceutical composition comprises about 1,008 mg of an FcRn antagonist. In some embodiments, the pharmaceutical composition comprises 1,008 mg of an FcRn antagonist. In some embodiments, the FcRn antagonist is efgartigimod, or a biosimilar version thereof.

For SC administration, in certain embodiments efgartigimod may be administered co-formulated with hyaluronidase, for example, in particular, rHuPH20 (recombinant Human PH20). The co-formulated material will allow dosing of higher volumes.

rHuPH20 is the active ingredient of Halozyme's commercial product HYLENEX® recombinant (hyaluronidase human injection), referred to as HYLENEX®, which was approved by FDA for marketed use in the U.S. in December 2005. HYLENEX® is a tissue permeability modifier indicated as an adjuvant in SC fluid administration for achieving hydration, to increase the dispersion and absorption of other injected drugs, and in SC urography, for improving resorption of radiopaque agents.

rHuPH20 is a recombinant enzyme human hyaluronidase produced by genetically engineered Chinese hamster ovary (CHO) cells containing a deoxyribonucleic plasmid encoding a soluble fragment of human hyaluronidase (posterior head protein 20 [PH20]).

The HZ202 rHuPH20 DS is currently registered in HYLENEX® and other biologic drug products co-formulated with rHuPH20 DS. As such, in certain embodiments HZ202 rHuPH20 DS is used in the efgartigimod/rHuPH20 co-formulated product for SC administration (i.e., efgartigimod PH20 SC).

Provided in the co-formulations, combinations, uses and methods herein are soluble hyaluronidases. Soluble hyaluronidases include any that, upon expression, are secreted from a cell and exist in soluble form. Such soluble hyaluronidases include, but are not limited to, bacterial soluble hyaluronidases, non-human soluble hyaluronidases, such as bovine PH20 and ovine PH20, human soluble PH20, and variants thereof. Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g., DG44 CHO cells).

In some embodiments, rHuPH20 refers to the composition produced upon expression in a cell, such as a CHO cell, of nucleic acid encoding residues 36-482 of SEQ ID NO: 24, generally linked to the native or a heterologous signal sequence (residues 1-35 of SEQ ID NO: 24). rHuPH20 is produced by expression of a nucleic acid molecule, such as encoding amino acids 1-482 (set forth in SEQ ID NO: 24) in a mammalian cell. Translational processing removes the 35 amino acid signal sequence. As produced in the culture medium there is heterogeneity at the C-terminus such that the product, designated rHuPH20, includes a mixture of species that can include any one or more of the polypeptides 36-480, 36-481, and 36-482 of SEQ ID NO: 24, and some shorter polypeptides, in various abundance. Typically, rHuPH20 is produced in cells that facilitate correct N-glycosylation to retain activity, such as CHO cells (e.g., DG44 CHO cells). In some embodiments, one of the most abundant species is the 446 amino acid polypeptide corresponding to residues 36-481 of SEQ ID NO: 24. In some embodiments, rHuPH20 refers to polypeptides that are soluble or secreted upon expression in a mammalian cell and have at least 90%, 91%, 92%, 93%, 94%, 95%, 96%, 97%, 98%, 99%, or more sequence identity with residues 36-482 of SEQ ID NO: 24. In some embodiments, the rHuPH20 is the 447 amino acid polypeptide of SEQ ID NO: 25.

TABLE 3
Exemplary hyaluronidase sequences

| SEQ ID NO: | Amino Acid Sequence |
|---|---|
| 24 | MGVLKFKHIFFRSFVKSSGVSQIVFTFLLIPCCLTLNFRAPPVIPNVPF |
| | LWAWNAPSEFCLGKFDEPLDMSLFSFIGSPRINATGQGVTIFYVDRL |
| | GYYPYIDSITGVTVNGGIPQKISLQDHLDKAKKDITFYMPVDNLGM |
| | AVIDWEEWRPTWARNWKPKDVYKNRSIELVQQQNVQLSLTEATE |
| | KAKQEFEKAGKDFLVETIKLGKLLRPNHLWGYYLFPDCYNHHYKK |
| | PGYNGSCFNVEIKRNDDLSWLWNESTALYPSIYLNTQQSPVAATLY |
| | VRNRVREAIRVSKIPDAKSPLPVFAYTRIVFTDQVLKFLSQDELVYT |
| | FGETVALGASGIVIWGTLSIMRSMKSCLLLDNYMETILNPYIINVTL |
| | AAKMCSQVLCQEQGVCIRKNWNSSDYLHLNPDNFAIQLEKGGKFT |
| | VRGKPTLEDLEQFSEKFYCSCYSTLSCKEKADVKDTDAVDVCIADG |
| | VCIDAFLKPPMETEEPQIFYNASPSTLSATMFIVSILFLIISSVASL |
| 25 | LNFRAPPVIPNVPFLWAWNAPSEFCLGKFDEPLDMSLFSFIGSPRIN |
| | ATGQGVTIFYVDRLGYYPYIDSITGVTVNGGIPQKISLQDHLDKAK |
| | KDITFYMPVDNLGMAVIDWEEWRPTWARNWKPKDVYKNRSIELV |
| | QQQNVQLSLTEATEKAKQEFEKAGKDFLVETIKLGKLLRPNHLWG |
| | YYLFPDCYNHHYKKPGYNGSCPNVEIKRNDDLSWLWNESTALYPS |
| | IYLNTQQSPVAATLYVRNRVREAIRVSKIPDAKSPLPVFAYTRIVFT |
| | DQVLKFLSQDELVYTFGETVALGASGIVIWGTLSIMRSMKSCLLLD |
| | NYMETILNPYIINVTLAAKMCSQVLCQEQGVCIRKNWNSSDYLHL |
| | NPDNFAIQLEKGGKFTVRGKPTLEDLEQFSEKFYCSCYSTLSCKEK |
| | ADVKDTDAVDVCIADGVCIDAFLKPPMETEEPQIFY |

In some embodiments, the endoglycosidase hydrolase enzyme cleaves hyaluronic acid at a hexosaminidic β (1-4) or (1-3) linkage. In some embodiments, the endoglycosidase hydrolase enzyme comprises a catalytic domain of hyaluronidase PH-20 (HuPH20), HYAL1, HYAL2, HYAL3, HYAL4, or HYALPS1. In some embodiments, the endoglycosidase hydrolase enzyme comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to amino acids 36-490 of SEQ ID NO: 32. In some embodiments, the endoglycosidase hydrolase enzyme comprises a hyaluronidase. In some embodiments, the endoglycosidase hydrolase enzyme comprises a hyaluronidase selected from the group consisting of HuPH20, HYALI, HYAL2, HYAL3, HYAL4, any variant, and any isoform thereof. In some embodiments, the endoglycosidase hydrolase enzyme comprises rHuPH20 or a fragment thereof.

In some embodiments, the endoglycosidase hydrolase enzyme comprises a modified hyaluronidase comprising one or more amino acid substitutions relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase comprising one or more amino acid substitution in an alpha-helix region relative to a wild-type hyaluronidase selected from the group consisting of HuPH20, HYALI, HYAL2, HYAL3, HYAL4, HYALPS1, or a fragment thereof. In some embodiments, the endoglycosidase hydrolase enzyme comprises a modified hyaluronidase comprising one or more amino acid substitution in linker region relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase, wherein one or more N-terminal and/or C-terminal amino acids are deleted relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified rHuPH20, wherein the modified rHuPH20 comprises: i. one or more amino acid substitution in an alpha-helix region, a linker region, or both an alpha-helix region and a linker region relative to wild-type rHuPH20; ii. deletion of one or more N-terminal amino acid, one or more C-terminal amino acid, or one or more N-terminal amino acid and one or more C-terminal amino acid relative to wild-type rHuPH20; or iii. both (i) and (ii).

“Hyaluronidase,” as used herein, refers to an enzyme capable of catalyzing the cleavage of hyaluronan. Hyaluronan is a repeating polymer of N-acetyl-glucosamine and glucuronic acid, which is present in the subcutaneous space and contributes to the soluble gel-like component of the extracellular matrix of the skin and is restored by rapid turnover (resynthesis). In some embodiments, the hyaluronidase comprises rHuPH20, which is a glycosylated 447-amino acid single chain polypeptide that depolymerizes hyaluronan in the subcutaneous space locally at the site of injection in the skin. Depolymerization of hyaluronan by hyaluronidase is accomplished by hydrolysis of the polysaccharide polymer. Depolymerization of hyaluronan results in a transient reduction in the viscosity of the gel-like phase of the extracellular matrix and increased hydraulic conductance that facilitates the dispersion and absorption of the coadministered therapeutic agent. Thus, a hyaluronidase, e.g., rHuPH20, can improve the speed and ease of subcutaneous delivery of injectable biologics and drugs by acting as a permeation enhancer. In certain embodiments, the hyaluronidase comprises ENHANZE™.

In some embodiments, the pharmaceutical composition comprises about 180 mg/mL of an FcRn antagonist, about 2,000 U/mL rHuPH20, about 1.4 mg/mL L-histidine, about 2.2 mg/mL L-histidine hydrochloride monohydrate, about 1.5 mg/mL L-methionine, about 0.4 mg/mL polysorbate 20, about 5.8 mg/mL sodium chloride, and about 20.5 mg/mL sucrose, at a pH of about 6.0. In some embodiments, the FcRn antagonist is efgartigimod, or a biosimilar version thereof.

In some embodiments, the pharmaceutical composition comprises 180 mg/ml of an FcRn antagonist, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, and 20.5 mg/mL sucrose, at a pH of 6.0. In some embodiments, the FcRn antagonist is efgartigimod, or a biosimilar version thereof.

In some embodiments, the pharmaceutical composition comprises 180 mg/mL efgartigimod, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, and 20.5 mg/mL sucrose, at a pH of 6.0.

In some embodiments, the pharmaceutical composition comprises about 180 mg/mL of an FcRn antagonist, about 2,000 U/mL rHuPH20, about 20 mM L-histidine/L-histidine hydrochloride, about 100 mM sodium chloride, about 60 mM sucrose, about 10 mM L-methionine, and about 0.04% (w/) polysorbate 20, at a pH of about 6.0. In some embodiments, the FcRn antagonist is efgartigimod, or a biosimilar version thereof.

In some embodiments, the pharmaceutical composition comprises 180 mg/mL of an FcRn antagonist, 2,000 U/mL rHuPH20, 20 mM L-histidine/L-histidine hydrochloride, 100 mM sodium chloride, 60 mM sucrose, 10 mM L-methionine, and 0.04% (w/) polysorbate 20, at a pH of 6.0. In some embodiments, the FcRn antagonist is efgartigimod, or a biosimilar version thereof.

In some embodiments, the pharmaceutical composition comprises 0.02-0.06% w/v polysorbate 20.

In some embodiments, the pH of the pharmaceutical composition is 6.0±0.3.

In any of the above embodiments, the pharmaceutical composition may be a unit dosage form.

In an embodiment, the unit dosage form comprises the FcRn antagonist as a dry formulation for dissolution such as a lyophilized powder, freeze-dried powder, or water-free concentrate. In an embodiment, the dry formulation is comprised in a hermetically sealed container such as a vial, an ampoule, or a sachet.

In an embodiment, the unit dosage form comprises the FcRn antagonist as a liquid formulation, e.g., injection solution. In an embodiment, the liquid formulation is comprised in a hermetically sealed container such as a vial. In an embodiment, the liquid formulation is comprised in a single-dose vial.

In some embodiments, the dosage form is overfilled to allow withdrawal of a given amount of formulation. In some embodiments, the dosage form is a single-dose vial that contains at least 5.6 mL of a liquid formulation comprising an FcRn antagonist at a concentration of 180 mg/mL. In some embodiments the vial is overfilled. In some embodiments, the overfilled vial comprises 5.6-6.9, 5.6-6.5, 5.6-6.0, or 5.6-5.8 mL of liquid formulation. In some embodiments, the overfilled vial comprises about 5.6, about 5.7, about 5.8, about 5.9, about 6.0, about 6.1, about 6.2, about 6.3, about 6.4, about 6.5, about 6.6, about 6.7, about 6.8, or about 6.9 mL of liquid formulation. In some embodiments, the overfilled vial comprises 5.6, 5.7, 5.8, 5.9, 6.0, 6.1, 6.2, 6.3, 6.4, 6.5, 6.6, 6.7, 6.8, or 6.9 mL of liquid formulation. In some embodiments, the overfilled vial comprises about 5.8 mL of liquid formulation. In some embodiments, the overfilled vial comprises 5.8 mL of liquid formulation. In some embodiments, the liquid formulation comprises efgartigimod, or a biosimilar version thereof. In some embodiments, the liquid formulation comprises efgartigimod and rHuPH20.

In an embodiment, the single-dose vial may contain 5.6 mL of a liquid formulation comprising an FcRn antagonist in an amount of 1,008 mg. In some embodiments, the single-dose vial is overfilled to allow withdrawal of 5.6 mL or more of the liquid formulation from the vial. In some embodiments, the single-dose vial is overfilled by 2-5%. In some embodiments, the single-dose vial is overfilled by 3%. In some embodiments, the single-dose vial contains 5.8 mL of a liquid formulation comprising an FcRn antagonist in an amount of 1044 mg. In some embodiments, the FcRn antagonist is efgartigimod, or a biosimilar version thereof.

In some embodiments, the single-dose vial contains 5.6 mL of a liquid formulation comprising 1,008 mg of an FcRn antagonist, and 11,200 units rHuPH20. Each mL of vial solution contains 180 mg of efgartigimod alfa, 2,000 units of hyaluronidase (human recombinant) and histidine (1.4 mg), L-histidine hydrochloride monohydrate (2.2 mg), methionine (1.5 mg), polysorbate 20 (0.4 mg), sodium chloride (5.8 mg), sucrose (20.5 mg), and water for injection, USP, at a pH of 6.0. In some embodiments, the FcRn antagonist is efgartigimod, or a biosimilar version thereof.

In some embodiments, the liquid formulation has a viscosity at room temperature of 4-8 mPa s. In some embodiments, the liquid formulation has a viscosity at room temperature of 4-7 mPa s, 5-7 mPa s, 4-6 mPa s, or 5-6 mPa s. In some embodiments, the liquid formulation has a viscosity at room temperature of about 4 mPa s, about 5 mPa s, about 6 mPa s, about 7 mPa s, or about 8 mPa s.

In some embodiments, the pharmaceutical composition is a sterile, preservative free, yellowish, clear to opalescent solution supplied in a single-dose vial for subcutaneous injection.

In some embodiments, the pharmaceutical compositions described herein are contraindicated in patients with serious hypersensitivity to the FcRn antagonist (e.g., efgartigimod), to hyaluronidase, or to any of the excipients in the compositions. In some embodiments, the hypersensitivity is anaphylaxis and/or hypotension leading to syncope.

### Kits

The present disclosure provides kits comprising an FcRn antagonist and a label. In some embodiments, the kit comprises a pharmaceutical composition comprising an FcRn antagonist and a label. Any FcRn antagonist described herein, or pharmaceutical composition thereof, may be included in the kits provided herein. In some embodiments, the FcRn antagonist is efgartigimod, or a biosimilar version thereof.

In some embodiments, the kit comprises a single-dose vial comprising the FcRn antagonist as a liquid formulation, and a label.

In some embodiments, each single-dose vial is provided in an individual carton to protect the vial from light until time of use. In some embodiments, the carton is provided with at least one copy of instructions for use.

In some embodiments, the single-dose vial contains 5.6 mL of a liquid formulation comprising 1,008 mg of an FcRn antagonist, and 11,200 units rHuPH20. Each mL of vial solution contains 180 mg of efgartigimod alfa, 2,000 units of hyaluronidase (human recombinant) and histidine (1.4 mg), L-histidine hydrochloride monohydrate (2.2 mg), methionine (1.5 mg), polysorbate 20 (0.4 mg), sodium chloride (5.8 mg), sucrose (20.5 mg), and water for injection, USP, at a pH of 6.0. In some embodiments, the FcRn antagonist is efgartigimod, or a biosimilar version thereof.

In some embodiments, the instructions are provided on a label. In some embodiments, the instructions provide information regarding storage of the single-dose vial. In some embodiments, the information provides that the single-dose vial should be stored in the refrigerator between 36° F. to 46° F. (2° C. to 8° C.). In some embodiments, the information provides that the single-dose vials should be stored in the original carton to protect them from light until time of use. In some embodiments, the information provides that the single-dose vials should not be frozen. In some embodiments, the information provides that the single-dose vials should not be shaken. In some embodiments, the instructions provide that unopened vials may be stored in the original carton for up to 3 days at room temperature at 68° F. to 77° F. (20° C. to 25° C.) for a single period before administration or returned to refrigeration. In some embodiments, the instructions provide that the single-dose vial should not be stored at room temperature more than one time. In some embodiments, the instructions provide to record the date removed from and the date returned to the refrigerator on the carton.

In some embodiments, the instructions provide steps for preparing for the injection. In some embodiments, the steps include taking the single-dose vial out of the refrigerator at least 15 minutes before injecting to allow it to warm to room temperature. No external heat sources should be used. In some embodiments, the steps include checking that the solution in the single-dose vial is yellowish, clear to opalescent. In some embodiments, the steps include inspecting visually for particulate matter prior to administration, whenever solution and container permit. In some embodiment, the steps indicated that the single-dose vial should not be used if opaque particles or other foreign particles are present. In some embodiments, the steps include withdrawing the entire content of the solution from the single-dose vial using a polypropylene syringe and an 18G stainless steel transfer needle. In some embodiments, the steps include removing large air bubbles, if present. In some embodiments, the steps indicate that each vial contains overfill to compensate for liquid loss during preparation and to compensate for the priming volume of the winged infusion set. In some embodiments, the steps indicate to administer immediately after preparation.

In some embodiments, the information provides steps for administering the injection. In some embodiments, the steps indicate to administer the solution using a winged infusion set made of polyvinyl chloride (PVC), 25G, 12 inches tubing, maximum priming volume of 0.4 mL. In some embodiments, the steps include removing the transfer needly from the syringe and connecting the syringe to the winged infusion set. In some embodiments, the steps include filling the tubing of the winged infusion set prior to administration by gently pressing the syringe plunger until the plunger is at 5.6 mL. In some embodiments, the steps indicate that there should be solution at the end of the winged infusion set needle. In some embodiments, the steps include choosing an injection site on the abdomen at least two inches away from the navel. The injection site should be rotated (i.e., changed) for each injection. The injection should not be performed on skin that is red, bruised, tender, hard, or into areas where there are moles or scars. In some embodiments, the steps include injecting the solution subcutaneously into a pinched skin area at an angle of about 45° over 30 to 90 seconds. In some embodiments, the steps indicate to discard any unused portions of medicine remaining in the vial, the syringe, and the winged infusion set. In some embodiments, the steps indicate that localized injection site reactions may occur after administration. In some embodiments, the steps indicate that healthcare professionals should monitor for clinical signs and symptoms of hypersensitivity reactions for at least 30 minutes after administration. If a hypersensitivity reaction occurs, the healthcare professional should institute appropriate measures if needed or the patient should seek medical attention.

In some embodiments, the label includes an indication for the treatment of gMG in adult patients who are anti-AChR antibody positive. In some embodiments, the label includes an indication for the treatment of adults with gMG who do not have sufficient response to steroids or non-steroidal immunosuppressive therapies (ISTs). In some embodiments, the label includes data demonstrating an MG-ADL response rate of 67.7% and a QMG response rate of 63.1% in a population of gMG patients who received 10 mg/kg efgartigimod, compared to a MG-ADL response rate of 29.7% and a QMG response rate of 14.1% in a population of gMG patients who received placebo.

In some embodiments, the label includes a contraindication in patients with serious hypersensitivity to efgartigimod alfa products, to hyaluronidase, or to any excipients in formulations thereof. In some embodiments, the label includes a warning for hypersensitivity reactions selected from anaphylaxis and hypotension leading to syncope. In some embodiments, the label states infusion discontinuation when anaphylaxis or hypotension leading to syncope occur during or within 1 hour of administration of the product. In some embodiments, the label includes a warning for infusion-related reactions selected from hypertension, chills, shivering, thoracic pain, abdominal pain, and back pain. In some embodiments, the label states to discontinue infusion and initiate appropriate therapy when a severe infusion-related reaction occurs during administration of the product. In some embodiments, the label states that patients may be rechallenged with close clinical observation, slower infusion rates, and pre-medications when a mild-to-moderate infusion-related reaction occurs during administration of the product. In some embodiments, the label includes a combination of any of the features described above or elsewhere herein.

### Methods

The present disclosure provides methods of reducing serum IgG levels in a subject in need thereof comprising administering to the subject an effective amount of an FcRn antagonist according to the disclosure or a pharmaceutical composition comprising the same. The disclosure also provides methods for treating an antibody-mediated disorder (e.g., an autoantibody-mediated disorder) in a subject comprising administering to the subject an effective of an FcRn antagonist according to the disclosure or a pharmaceutical composition comprising the same. In some embodiments, the antibody-mediated disorder is an autoimmune disease. In some embodiments, the autoimmune disease is myasthenia gravis (e.g., gMG).

Approximately 80% of all MG patients, but only about 50% of ocular MG (oMG) patients, test positive for the presence of anti-AChR autoantibodies (i.e., are anti-AChR antibody positive). AChR autoantibodies can be measured using one or more art-recognized methods, including radioimmunoprecipitation and cell-based assays (see Jacob et al., Arch. Neurol., 2012; 69:994-1001), as well as ELISA and fluorescence assays based on immunoprecipitation (see Yang et al., J Neurol. Sci., 2011; 301:71-76). In some embodiments, the subject has gMG and is anti-AChR antibody positive. In some embodiments, the subject has gMG and is anti-AChR antibody negative. In some embodiments, the subject has gMG and does not have sufficient response to steroids or non-steroidal immunosuppressive therapies as determined by a health care provider.

The present disclosure provides methods of subcutaneously administering an FcRn antagonist. In some embodiments, the FcRn antagonist is provided in a pharmaceutical composition in a single-dose vial. In some embodiments, the single-dose vial is stored in a refrigerator. In some embodiments, the single-dose vial is stored at a temperature of 2° C.-8° C. In some embodiments, the single-dose vial is warmed to room temperature before being administered to a subject. In some embodiments, the single-dose vial is warmed to 20° C.-25° C. before being administered to a subject. In some embodiments, the single-dose vial is warmed to about 20, about 21, about 22, about 23, about 24, or about 25° C. before being administered to a subject. In some embodiments, the single-dose vial is warmed to 20, 21, 22, 23, 24, or 25° C. before being administered to a subject. In some embodiments, the single-dose vial is warmed for at least 15 minutes. In some embodiments, the single-dose vial is warmed for about 15, about 20, about 25, about 30, about 35, about 40, about 45, about 50, about 55, or about 60 minutes. In some embodiments, the single-dose vial is warmed for 15, 20, 25, 30, 35, 40, 45, 50, 55, or 60 minutes. In some embodiments, the single-dose vial is stored in its original container. In some embodiments, the single-dose vial should not be frozen when stored. In some embodiments, the single-dose vial should not be shaken when stored.

In some embodiments, the contents of the single-dose vial are withdrawn using a polypropylene syringe and an 18G stainless steel transfer needle. In some embodiments, large air bubbles are removed from the syringe, if present. In some embodiments, the FcRn antagonist is administered immediately after preparation.

In some embodiments, the FcRn antagonist is administered using a winged infusion set. In some embodiments, the winged infusion set is made of PVC, 25G, 12 inches tubing, maximum priming volume of 0.4 mL. In some embodiments, the transfer needle is removed from the syringe and the syringe is connected to the winged infusion set. In some embodiments, prior to administration, the tubing of the winged infusion set is filled by gently pressing the syringe plunger until the plunger is at 5.6 mL. In some embodiments, there should be solution at the end of the winged infusion set needle. In some embodiments, the injection site is chosen on the abdomen of the subject. In some embodiments, the injection site is within 2-4 inches of the navel of the subject. In some embodiments, the injection site is within 2-3 inches of the navel of the subject. In some embodiments, the injection site is within about 0.5, about 1.0, about 1.5, about 2.0, about 2.5, about 3.0, about 3.5, or about 4.0 inches of the navel of the subject. In some embodiments, the injection site is within 0.5, 1.0, 1.5, 2.0, 2.5, 3.0, 3.5, or 4.0 inches of the navel of the subject.

In some embodiments, the needle is inserted into the injection site. In some embodiments, the needle is inserted into the injection site while it is pinched. In some embodiments, the needle is inserted into the injection site at about a 45° angle to the surface of the injection site.

In some embodiments, the FcRn antagonist is administered over about 30 to 90 seconds. In some embodiments, the FcRn antagonist is administered over 30 to 90 seconds. In some embodiments, the liquid pharmaceutical composition is at room temperature during administration. In some embodiments, the liquid pharmaceutical composition is at 20° C.-25° C. during administration.

In some embodiments, the administration is performed by a healthcare professional to the subject.

### Dosing

In some embodiments, the FcRn antagonist is administered at a fixed dose of about 20 mg to about 20,000 mg. In some embodiments, the FcRn antagonist is administered at a fixed dose of about 200 mg to about 20,000 mg. In some embodiments, the FcRn antagonist is administered at a fixed dose of about 300 mg to about 6000 mg. In some embodiments, the FcRn antagonist is administered at a fixed dose of about 750 mg to about 3000 mg. In some embodiments, the FcRn antagonist is administered at a fixed dose of about 1000 mg to about 2500 mg. In some embodiments, the FcRn antagonist is administered at a fixed dose of about 1000 mg to about 2000 mg. In some embodiments, the FcRn antagonist is administered at a fixed dose of about 1000 mg. In some embodiments, the FcRn antagonist is administered at a fixed dose of about 1008 mg. In some embodiments, the FcRn antagonist is efgartigimod, or a biosimilar version thereof.

In some embodiments, the FcRn antagonist is administered at a fixed dose of 20 mg to 20,000 mg. In some embodiments, the FcRn antagonist is administered at a fixed dose of 200 mg to 20,000 mg. In some embodiments, the FcRn antagonist is administered at a fixed dose of 300 mg to 6000 mg. In some embodiments, the FcRn antagonist is administered at a fixed dose of 750 mg to 3000 mg. In some embodiments, the FcRn antagonist is administered at a fixed dose of 1000 mg to 2500 mg. In some embodiments, the FcRn antagonist is administered at a fixed dose of 1000 mg to 2000 mg. In some embodiments, the FcRn antagonist is administered at a fixed dose of 1000 mg. In some embodiments, the FcRn antagonist is administered at a fixed dose of 1008 mg. In some embodiments, the FcRn antagonist is efgartigimod, or a biosimilar version thereof.

In some embodiments, the FcRn antagonist is administered at a fixed dose of about 20 mg, about 50 mg, about 100 mg, about 200 mg, about 250 mg, about 300 mg, about 500 mg, about 750 mg, about 1000 mg, about 1008 mg, about 1200 mg, about 1500 mg, about 2000 mg, about 2500 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg. In some embodiments, the FcRn antagonist is efgartigimod, or a biosimilar version thereof.

In some embodiments, the FcRn antagonist is administered at a fixed dose of 20 mg, 50 mg, 100 mg, 200 mg, 250 mg, 300 mg, 500 mg, 750 mg, 1000 mg, 1008 mg, 1200 mg, 1500 mg, 2000 mg, 2500 mg, 3000 mg, 4000 mg, 5000 mg, 6000 mg, 7000 mg, 8000 mg, 9000 mg, 10,000 mg, 11,000 mg, 12,000 mg, 13,000 mg, 14,000 mg, 15,000 mg, 16,000 mg, 17,000 mg, 18,000 mg, 19,000 mg, or 20,000 mg. In some embodiments, the FcRn antagonist is efgartigimod, or a biosimilar version thereof.

In some embodiments, the FcRn antagonist is administered at a dose of about 0.2 mg/kg to about 200 mg/kg. In some embodiments, the FcRn antagonist is administered at a dose of about 2 mg/kg to about 200 mg/kg. In some embodiments, the FcRn antagonist is administered at a dose of about 2 mg/kg to about 120 mg/kg. In some embodiments, the FcRn antagonist is administered at a dose of about 3 mg/kg to about 60 mg/kg. In some embodiments, the FcRn antagonist is administered at a dose of about 10 mg/kg to about 25 mg/kg. In some embodiments, the FcRn antagonist is efgartigimod, or a biosimilar version thereof.

In some embodiments, the FcRn antagonist is administered at a dose of 0.2 mg/kg to 200 mg/kg. In some embodiments, the FcRn antagonist is administered at a dose of about 2 mg/kg to about 200 mg/kg. In some embodiments, the FcRn antagonist is administered at a dose of 2 mg/kg to 120 mg/kg. In some embodiments, the FcRn antagonist is administered at a dose of 3 mg/kg to 60 mg/kg. In some embodiments, the FcRn antagonist is administered at a dose of 10 mg/kg to 25 mg/kg. In some embodiments, the FcRn antagonist is efgartigimod, or a biosimilar version thereof.

In some embodiments, the FcRn antagonist is administered at a dose of about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the FcRn antagonist is efgartigimod, or a biosimilar version thereof.

In some embodiments, the FcRn antagonist is administered at a dose of 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the FcRn antagonist is efgartigimod, or a biosimilar version thereof.

In some embodiments, the FcRn antagonist is administered subcutaneously. In some embodiments, the FcRn antagonist is administered subcutaneously once weekly, once every 2 weeks, once every 3 weeks, once every 4 weeks, once monthly, or once every 6 weeks. In some embodiments, the FcRn antagonist is efgartigimod, or a biosimilar version thereof.

In some embodiments, the FcRn antagonist is administered subcutaneously at fixed dose of 100 mg to 10,000 mg once weekly, once every 2 weeks, once every 3 weeks, once every 4 weeks, once monthly, or once every 6 weeks. In some embodiments, the FcRn antagonist is administered subcutaneously at fixed dose of 1000 mg or 2000 mg once weekly, once every 2 weeks, once every 3 weeks, once every 4 weeks, once monthly, or once every 6 weeks. In some embodiments, the FcRn antagonist is efgartigimod, or a biosimilar version thereof.

In some embodiments, the FcRn antagonist is administered using a phased dosing schedule comprising multiple treatment cycles. In some embodiments, the phased dosing schedule comprises 2, 3, 4, 5, 6, 7, 8, 9, 10, or more treatment cycles. In some embodiments, each treatment cycle independently comprises or consists of administration of 1-5 doses of the FcRn antagonist within 1 month. In some embodiments, at least one treatment cycle comprises or consists of weekly administration of the FcRn antagonist for 4 weeks. In some embodiments, each treatment cycle comprises or consists of weekly administration of the FcRn antagonist for 4 weeks. In some embodiments, the FcRn antagonist is efgartigimod, or a biosimilar version thereof.

In some embodiments, the phased dosing schedule comprises a first treatment cycle and one or more subsequent treatment cycles. In some embodiments, the first treatment cycle and the one or more treatment cycles each independently comprise or consist of administration of 1-5 doses of the FcRn antagonist within 1 month. In some embodiments, the first treatment cycle comprises or consists of weekly administration of the FcRn antagonist for 4 weeks. In some embodiments, at least one of the one or more subsequent treatment cycles comprises or consists of weekly administration of the FcRn antagonist for 4 weeks. In some embodiments, each of the one or more subsequent treatment cycles comprise or consist of weekly administration of the FcRn antagonist for 4 weeks. In some embodiments, each of the one or more subsequent treatment cycles is administered to the subject based on clinical evaluation by a health care provider. In some embodiments, each of the one or more subsequent treatment cycles is administered ≥50 days from the start of the previous treatment cycle. In some embodiments, the FcRn antagonist is efgartigimod, or a biosimilar version thereof.

In some embodiments, treatment according to any of the dosing regimens described herein is followed by a period wherein the subject does not receive any further FcRn antagonist. Subjects receiving an FcRn antagonist according to any of the dosing regimens described herein may achieve clinical response such that treatment can be withdrawn. In some embodiments, clinical response is defined as MG-ADL response, QMG response, or both MG-ADL response and QMG response. The subject may remain untreated during a period wherein clinical response has been achieved, optionally with a period of further treatment if clinical deterioration occurs. In some embodiments, clinical deterioration refers to new or worsening respiratory/bulbar symptoms or at least a 2-point increase of individual non-ocular MG-ADL items. If, further to treatment according to any of the dosing regimens described herein, clinical response is not achieved or relapse occurs, the subject may undergo a further period of treatment with the FcRn antagonist in accordance with any of the dosing regimens described herein. In some embodiments, a subsequent treatment cycle is administered when the subject exhibits one or more of the following: a total MG-ADL score of ≥5; a total MG-ADL score of ≥5 with more than 50% of the score due to non-ocular symptoms; a loss of MG-ADL response; and a reduction of total MG-ADL score of <2 points compared to the score at the previous treatment cycle baseline. In some embodiments, loss of MG-ADL response is defined as no longer showing a decrease of ≥2 points on the total MG-ADL score compared to the corresponding treatment cycle baseline. In some embodiments, the FcRn antagonist is efgartigimod, or a biosimilar version thereof.

### Hypersensitivity and Infusion/Injection-Related Reactions

Efgartigimod, an FcRn antagonist according to the instant disclosure, was approved by the FDA in 2021 for the treatment of gMG in adult patients who are anti-AChR antibody positive. VYVGART HYTRULO was approved by the FDA in June 2023 for the treatment of gMG in adult patients who are anti-AChR antibody positive. Approval was based upon data establishing the efficacy of efgartigimod in adult patients with gMG who are anti-AChR antibody positive in clinical trial NCT03669588. A total of 167 patients were enrolled in NCT03669588 and were randomized to receive either VYVGART (efgartigimod alfa-fcab) 10 mg/kg (1200 mg for those weighing 120 kg or more) (n=84) or placebo (n=83). Baseline characteristics were similar between treatment groups. Patients had a median age of 46 years at screening (range: 19 to 81 years) and a median time since diagnosis of 9 years. Seventy-one percent were female, and 84% were White. Median MG-ADL total score was 9, and median Quantitative Myasthenia Gravis (QMG) total score was 16. The majority of patients (n=65 for VYVGART; n=64 for placebo) were positive for AChR antibodies.

At baseline, over 80% of patients in each group received AChE inhibitors, over 70% in each treatment group received steroids, and approximately 60% in each treatment group received NSISTs, at stable doses.

Patients were treated with VYVGART at the recommended dosage regimen described above.

The efficacy of VYVGART was measured using the Myasthenia Gravis-Specific Activities of Daily Living scale (MG-ADL) which assesses the impact of gMG on daily functions of 8 signs or symptoms that are typically affected in gMG. Each item is assessed on a 4-point scale where a score of 0 represents normal function and a score of 3 represents loss of ability to perform that function. A total score ranges from 0 to 24, with the higher scores indicating more impairment. In this study, an MG-ADL responder was defined as a patient with a 2-point or greater reduction in the total MG-ADL score compared to the treatment cycle baseline for at least 4 consecutive weeks, with the first reduction occurring no later than 1 week after the last injection of the cycle.

The primary efficacy endpoint was the comparison of the percentage of MG-ADL responders during the first treatment cycle between treatment groups in the AChR-Ab positive population. A statistically significant difference favoring VYVGART was observed in the MG-ADL responder rate during the first treatment cycle [67.7% in the VYVGART-treated group vs 29.7% in the placebo-treated group (p<0.0001)].

The efficacy of VYVGART was also measured using the Quantitative Myasthenia Gravis (QMG) total score which is a 13-item categorical grading system that assesses muscle weakness. Each item is assessed on a 4-point scale where a score of 0 represents no weakness and a score of 3 represents severe weakness. A total possible score ranges from 0 to 39, where higher scores indicate more severe impairment. In this study, a QMG responder was defined as a patient who had a 3-point or greater reduction in the total QMG score compared to the treatment cycle baseline for at least 4 consecutive weeks, with the first reduction occurring no later than 1 week after last injection of the cycle.

The secondary endpoint was the comparison of the percentage of QMG responders during the first treatment cycle between both treatment groups in the AChR-Ab positive patients. A statistically significant difference favoring VYVGART was observed in the QMG responder rate during the first treatment cycle [63.1% in the VYVGART-treated group vs 14.1% in the placebo-treated group (p<0.0001)].

VYVGART HYTRULO was initially approved with no contraindications. Hypersensitivity reactions, including rash, angioedema, and dyspnea, were observed in the clinical trials. Urticaria was also observed in patients treated with VYVGART HYTRULO. These hypersensitivity reactions were mild-to-moderate, occurred within 1 hour to 3 weeks of administration, and did not lead to treatment discontinuation. No injection-related reactions were reported.

Since approval of VYVGART HYTRULO in 2023, the following unexpected new adverse reactions have been studied in patients receiving intravenous efgartigimod alfa-fcab: hypersensitivity reactions including anaphylaxis and hypotension, and administration-related reactions. Unexpectedly, anaphylaxis and hypotension leading to syncope occurred during or within an hour of administration and led to administration discontinuation and in some cases to permanent discontinuation.

Thus, in some embodiments, subjects treated in accordance with the methods described herein are monitored for a hypersensitivity reaction selected from the group consisting of anaphylaxis and hypotension leading to syncope, and appropriate measures are instituted if needed. In some embodiments, the subjects are monitored for at least 30 minutes after administration of the FcRn antagonist. In some embodiments, the FcRn antagonist is administered to the subject via subcutaneous administration over 30 to 90 seconds. In some embodiments, the appropriate measure to be initiated following detection of anaphylaxis and/or hypotension leading to syncope is discontinuation of the subcutaneous administration. In some embodiments, the appropriate measure to be initiated following detection of anaphylaxis and/or hypotension leading to syncope is permanent discontinuation of treatments with the FcRn antagonist. In some embodiments, the subject is informed of the signs and symptoms of a hypersensitivity reaction, such as anaphylaxis and/or hypotension leading to syncope, and is advised to contact a healthcare provider immediately should they occur. In some embodiments, the FcRn antagonist is efgartigimod, or a biosimilar version thereof.

Administration-related reactions reported with intravenous efgartigimod alfa-fcab following initial approval (i.e., postmarketing) include hypertension, chills, shivering, thoracic pain, abdominal pain, and back pain. Administration-related reactions occurred during or within an hour of administration and led to administration discontinuation.

Thus, in some embodiments, subjects treated in accordance with the methods described herein are monitored for an injection-related reaction, and the injection is discontinued if an injection-related reaction is detected. In some embodiments, the subjects are monitored during administration of the FcRn antagonist and for at least 30 minutes after administration. In some embodiments, the subjects are monitored during administration of the FcRn antagonist and for 1 hour after administration. In some embodiments, the subject is rechallenged with the FcRn antagonist when the injection-related reaction is mild-to-moderate as determined by a health care provider. In some embodiments, rechallenging comprises one or more of close clinical observation, slower injection rate, and pre-medication. In some embodiments, rechallenging comprises close clinical observation, slower administration rate, and pre-medication. In some embodiments, the injection is discontinued when the injection-related reaction is severe and appropriate therapy is initiated. In some embodiments, the FcRn antagonist is readministered following a severe injection-related reaction if the benefit is determined to justify the potential risk. In some embodiments, a determination of a mild-to-moderate injection-related reaction or a severe injection-related reaction is made by a health care provider based upon guidance in the Common Terminology Criteria for Adverse Events (CTCAE), Version 5.0, published Nov. 27, 2017 (evs.nci.nih.gov/ftp1/CTCAE/CTCAE_5.0/). In some embodiments, the injection-related reaction comprises one or more of the following: hypertension, chills, shivering, thoracic pain, abdominal pain, and back pain. In some embodiments, the subject is advised of the potential risk of an injection-related reaction. In some embodiments, the FcRn antagonist is efgartigimod, or a biosimilar version thereof.

In any of the above methods, the FcRn antagonist may be comprised in a pharmaceutical composition as described herein. In some embodiments, the pharmaceutical composition comprises 180 mg/mL of an FcRn antagonist, 2000 U/mL rHuPH20, 20 mM L-histidine/L-histidine hydrochloride, 100 mM sodium chloride, 60 mM sucrose, 10 mM L-methionine, and 0.04% (w/) polysorbate 20, at a pH of 6.0. In some embodiments, the FcRn antagonist is efgartigimod, or a biosimilar version thereof.

### Approved Products

The present disclosure also provides approved products containing efgartigimod for the treatment of gMG. Methods of treating gMG comprising administering an approved product containing efgartigimod are also provided. Use of an approved product containing efgartigimod for the treatment of gMG is also provided.

In some embodiments, the approved product is for subcutaneous administration.

In some embodiments, the approved product is provided in a single-dose vial containing 180 mg/mL efgartigimod, 2000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, 20.5 mg sucrose, and water for injection, USP, at a pH of 6.0.

In some embodiments, the product is approved for treatment of gMG in adult patients who are anti-AChR antibody positive. In some embodiments, the product is approved for treatment of gMG in adult patients who do not have sufficient response to steroids or non-steroidal immunosuppressive therapies.

In some embodiments, the approved product is a reference product.

In some embodiments, the label for the approved product includes a contraindication in patients with serious hypersensitivity to efgartigimod alfa products, to hyaluronidase, or to any excipients in formulations thereof. In some embodiments, the label for the approved product includes a warning for hypersensitivity reactions selected from anaphylaxis and hypotension leading to syncope. In some embodiments, the label for the approved product includes a warning for infusion-related reactions selected from hypertension, chills, shivering, thoracic pain, abdominal pain, and back pain. In some embodiments, the label for the approved product states to initiate appropriate therapy when a severe infusion-related reaction occurs. In some embodiments, the label of the approved product states that patients may be rechallenged with close clinical observation, slower infusion rates, and pre-medications when a mild-to-moderate infusion-related reaction occurs. In some embodiments, the label of the approved product includes data demonstrating an MG-ADL response rate of 67.7% and a QMG response rate of 63.1% in a population of patients who received 10 mg/kg efgartigimod, compared to a MG-ADL response rate of 29.7% and a QMG response rate of 14.1% in a population of patients who received placebo. In some embodiments, the label of the approved product includes a combination of any of the features described above or elsewhere herein.

Biosimilars and bioequivalents of the approved product described herein are also encompassed by the instant disclosure.

Methods of treating gMG comprising administering an approved product containing efgartigimod, a biosimilar of an approved product containing efgartigimod, or a bioequivalent of an approved product containing efgartigimod to a subject are also provided. Use of an approved product containing efgartigimod, a biosimilar of an approved product containing efgartigimod, or a bioequivalent of an approved product containing efgartigimod for the treatment of gMG in a subject is also provided.

In some embodiments, the approved product, biosimilar, or bioequivalent is administered to the subject once weekly. In some embodiments, the approved product, biosimilar, or bioequivalent is administered to the subject once weekly for 4 weeks.

In some embodiments, the approved product, biosimilar, or bioequivalent is administered to the subject using a phased dosing schedule comprising a first treatment cycle and one or more subsequent treatment cycles, wherein the first treatment cycle and the one or more subsequent treatment cycles each independently comprise administration of 1-5 doses of the approved product, biosimilar, or bioequivalent within 1 month. In some embodiments, the first treatment cycle comprises weekly administration of the approved product, biosimilar, or bioequivalent for 4 weeks. In some embodiments, the one or more subsequent treatment cycles each comprise weekly administration of the approved product, biosimilar, or bioequivalent for 4 weeks. In some embodiments, each of the one or more subsequent treatment cycles is administered to the subject based on clinical evaluation. In some embodiments, each of the one or more subsequent treatment cycles is administered to the subject ≥50 days from the start of the previous treatment cycle.

In some embodiments, treatment according to any of the dosing regimens described herein is followed by a period wherein the subject does not receive any further treatment with the approved product, biosimilar, or bioequivalent. Subjects receiving an approved product, biosimilar, or bioequivalent according to any of the dosing regimens described herein may achieve clinical response such that treatment can be withdrawn. In some embodiments, clinical response is defined as MG-ADL response, QMG response, or both MG-ADL response and QMG response. The subject may remain untreated during a period wherein clinical response has been achieved, optionally with a period of further treatment if clinical deterioration occurs. In some embodiments, clinical deterioration refers to new or worsening respiratory/bulbar symptoms or at least a 2-point increase of individual non-ocular MG-ADL items. If, further to treatment according to any of the dosing regimens described herein, clinical response is not achieved or relapse occurs, the subject may undergo a further period of treatment with the approved product, biosimilar, or bioequivalent in accordance with any of the dosing regimens described herein. In some embodiments, a subsequent treatment cycle is administered when the subject exhibits one or more of the following: a total MG-ADL score of ≥5; a total MG-ADL score of ≥5 with more than 50% of the score due to non-ocular symptoms; a loss of MG-ADL response; and a reduction of total MG-ADL score of <2 points compared to the score at the previous treatment cycle baseline. In some embodiments, loss of MG-ADL response is defined as no longer showing a decrease of ≥2 points on the total MG-ADL score compared to the corresponding treatment cycle baseline.

In some embodiments, the subject is anti-AChR antibody positive. In some embodiments, the subject does not have sufficient response to steroids or non-steroidal immunosuppressive therapies.

In some embodiments, the methods or uses described herein further comprise monitoring the subject for a hypersensitivity reaction selected from the group consisting of anaphylaxis and hypotension leading to syncope; and initiating an appropriate measure to mitigate the hypersensitivity reaction when detected.

In some embodiments, the methods or uses described herein further comprise monitoring the subject for an infusion-related reaction; and discontinuing the infusion when an infusion-related reaction is detected. In some embodiments, the infusion-related reaction comprises one or more of the following: hypertension, chills, shivering, thoracic pain, abdominal pain, and back pain.

In some embodiments, the subject is a human, such as, for example, an adult human.

### EXAMPLES

The invention will be further understood with reference to the following non-limiting examples.

### Example 1: Label with Prescribing Information

The text below is from the label with the prescribing information for VYVGART® HYTRULO (efgartigimod alfa and hyaluronidase-qvfc) injection, for subcutaneous use, as originally approved by Food and Drug Administration under BLA 761304 (Initial U.S. Approval: June 2023).

### Highlights of Prescribing Information

These highlights do not include all the information needed to use VYVGART HYTRULO safely and effectively. See full prescribing information for VYVGART HYTRULO.

VYVGART® HYTRULO (efgartigimod alfa and hyaluronidase-qvfc) injection, for subcutaneous use Initial U.S. Approval: 2023

### Indications and Usage

VYVGART HYTRULO is a combination of efgartigimod alfa, a neonatal Fc receptor blocker, and hyaluronidase, an endoglycosidase, indicated for the treatment of generalized myasthenia gravis (gMG) in adult patients who are anti-acetylcholine receptor (AChR) antibody positive. (1)

### Dosage and Administration

See Full Prescribing Information for instructions on dosage, preparation, and administration. (2.1, 2.2, 2.3, 2.4)

Evaluate the need to administer age-appropriate vaccines according to immunization guidelines before initiation of a new treatment cycle with VYVGART HYTRULO. (2.1)

Administer by a healthcare professional only. (2.2)

For subcutaneous use with a winged infusion set. (2.2)

The recommended dose is 1,008 mg/11,200 units (1,008 mg efgartigimod alfa and 11,200 units hyaluronidase) administered subcutaneously over approximately 30 to 90 seconds in cycles of once weekly injections for 4 weeks. (2.3)

Administer subsequent treatment cycles based on clinical evaluation; the safety of initiating subsequent cycles sooner than 50 days from the start of the previous treatment cycle has not been established. (2.3)

### Dosage Forms and Strengths

Injection: 1,008 mg efgartigimod alfa and 11,200 units hyaluronidase per 5.6 mL (180 mg/2,000 units per mL) in a single-dose vial. (3)

### Contraindications

None. (4)

### Warnings and Precautions

Infections: Delay administration of VYVGART HYTRULO to patients with an active infection. Monitor for signs and symptoms of infection in patients treated with VYVGART HYTRULO. If serious infection occurs, administer appropriate treatment and consider withholding VYVGART HYTRULO until the infection has resolved. (5.1)

Hypersensitivity reactions: angioedema, dyspnea, and rash have occurred in patients treated with VYVGART HYTRULO. If a hypersensitivity reaction occurs, the healthcare professional should institute appropriate supportive measures if needed or the patient should seek medical attention. (5.2)

### Adverse Reactions

The most common adverse reactions (≥10%) in patients with gMG treated with efgartigimod alfa-fcab were respiratory tract infections, headache, and urinary tract infection.

Additional common adverse reactions with VYVGART HYTRULO are injection site reactions. (6.1)

To report SUSPECTED ADVERSE REACTIONS, contact Argenx at 1-833-argx411 or FDA at 1-800-FDA-1088 or fda.gov/medwatch.

### Drug Interactions

Closely monitor for reduced effectiveness of medications that bind to the human neonatal Fc receptor. When concomitant long-term use of such medications is essential for patient care, consider discontinuing VYVGART HYTRULO and using alternative therapies. (7)

See 17 for PATIENT COUNSELING INFORMATION

### Full Prescribing Information: Contents*

1 INDICATIONS AND USAGE
2 DOSAGE AND ADMINISTRATION
2.1 Recommended Vaccination
2.2 Important Dosage and Administration Instructions
2.3 Recommended Dose and Dose Schedules
2.4 Preparation and Administration Instructions
3 DOSAGE FORMS AND STRENGTHS
4 CONTRAINDICATIONS
5 WARNINGS AND PRECAUTIONS
5.1 Infections
5.2 Hypersensitivity Reactions
6 ADVERSE REACTIONS
6.1 Clinical Trials Experience
7 DRUG INTERACTIONS
7.1 Effect of VYVGART HYTRULO on Other Drugs
8 USE IN SPECIFIC POPULATIONS
8.1 Pregnancy
8.2 Lactation
8.4 Pediatric Use
8.5 Geriatric Use
8.6 Renal Impairment
11 DESCRIPTION
12 CLINICAL PHARMACOLOGY
12.1 Mechanism of Action
12.2 Pharmacodynamics
12.3 Pharmacokinetics
12.6 Immunogenicity
13 NONCLINICAL TOXICOLOGY
13.1 Carcinogenesis, Mutagenesis, Impairment of Fertility
14 CLINICAL STUDIES
16 HOW SUPPLIED/STORAGE AND HANDLING
17 PATIENT COUNSELING INFORMATION
Sections or subsections omitted from the full prescribing information are not listed.

### Full Prescribing Information

### 1 Indications and Usage

VYVGART HYTRULO is indicated for the treatment of generalized myasthenia gravis (gMG) in adult patients who are anti-acetylcholine receptor (AChR) antibody positive.

### 2 Dosage and Administration

### 2.1 Recommended Vaccination

Because VYVGART HYTRULO causes transient reduction in IgG levels, immunization with live-attenuated or live vaccines is not recommended during treatment with VYVGART HYTRULO. Evaluate the need to administer age-appropriate immunizations according to immunization guidelines before initiation of a new treatment cycle with VYVGART HYTRULO [see Dosage and Administration (2.3) and Warnings and Precautions (5.1)].

### 2.2 Important Dosage and Administration Instructions

VYVGART HYTRULO is to be administered by a healthcare professional only.

VYVGART HYTRULO is for subcutaneous use only and administered with a winged infusion set [see Dosage and Administration (2.4)]. Do not administer intravenously.

Do not dilute VYVGART HYTRULO.

### 2.3 Recommended Dose and Dose Schedules

The recommended dosage of VYVGART HYTRULO is 1,008 mg/11,200 units (1,008 mg efgartigimod alfa and 11,200 units hyaluronidase) administered subcutaneously over approximately 30 to 90 seconds in cycles of once weekly injections for 4 weeks.

Administer subsequent treatment cycles according to clinical evaluation. The safety of initiating subsequent cycles sooner than 50 days from the start of the previous treatment cycle has not been established.

If a scheduled dose is missed, VYVGART HYTRULO may be administered up to 3 days after the scheduled time point. Thereafter, resume the original dosing schedule until the treatment cycle is completed.

### 2.4 Preparation and Administration Instructions

Use aseptic technique when preparing and administering VYVGART HYTRULO. Do not shake the vial. Each vial is for one time use only. Avoid exposure to direct sunlight.

### Preparation

Take the VYVGART HYTRULO vial out of the refrigerator at least 15 minutes before injecting to allow it to reach room temperature [see How Supplied/Storage and Handling (16)]. Do not use external heat sources.
Check that the VYVGART HYTRULO solution is yellowish, clear to opalescent.
Parenteral medicine products should be inspected visually for particulate matter prior to administration, whenever solution and container permit. Do not use if opaque particles or other foreign particles are present.
Withdraw the entire content of VYVGART HYTRULO from the vial using a polypropylene syringe and an 18G stainless steel transfer needle.
Remove large air bubbles, if present.
Each vial contains overfill to compensate for liquid loss during preparation and to compensate for the priming volume of the winged infusion set.
VYVGART HYTRULO does not contain preservatives. Administer immediately after preparation.

### Administration

To administer VYVGART HYTRULO use a winged infusion set made of polyvinyl chloride (PVC), 25G, 12 inches tubing, maximum priming volume of 0.4 mL.
Remove the transfer needle from the syringe and connect the syringe to the winged infusion set.
Prior to administration, fill the tubing of the winged infusion set by gently pressing the syringe plunger until the plunger is at 5.6 mL. There should be solution at the end of the winged infusion set needle.
Choose an injection site on the abdomen (at least 2 to 3 inches away from the navel).
Do not inject on areas where the skin is red, bruised, tender, hard, or into areas where there are moles or scars.
Rotate injection sites for subsequent administrations.
Inject VYVGART HYTRULO subcutaneously into a pinched skin area at an angle of about 45 degrees over 30 to 90 seconds.
Localized injection site reactions may occur after VYVGART HYTRULO is administered. [see Adverse Reactions (6.1)].
Discard any unused portions of medicine remaining in the vial, the syringe and the winged infusion set.
Healthcare professionals should monitor for clinical signs and symptoms of hypersensitivity reactions for at least 30 minutes after administration. If a hypersensitivity reaction occurs, the healthcare professional should institute appropriate measures if needed or the patient should seek medical attention [see Warnings and Precautions (5.2)].

### 3 Dosage Forms and Strengths

Injection: 1,008 mg efgartigimod alfa and 11,200 units hyaluronidase per 5.6 mL (180 mg/2,000 units per mL) as yellowish, clear to opalescent solution, in a single-dose vial.

### 4 Contraindications

None.

### 5 Warnings and Precautions

### 5.1 Infections

VYVGART HYTRULO may increase the risk of infection. The most common infections observed in Study 1 were urinary tract infection (10% of efgartigimod alfa-fcab-treated patients compared to 5% of placebo-treated patients) and respiratory tract infections (33% of efgartigimod alfa-fcab-treated patients compared to 29% of placebo-treated patients) [see Adverse Reactions (6.1) and Clinical Studies (14)]. A higher frequency of patients who received efgartigimod alfa-fcab compared to placebo were observed to have below normal levels for white blood cell counts (12% versus 5%, respectively), lymphocyte counts (28% versus 19%, respectively), and neutrophil counts (13% versus 6%, respectively). The majority of infections and hematologic abnormalities were mild to moderate in severity. Delay VYVGART HYTRULO administration in patients with an active infection until the infection is resolved. During treatment with VYVGART HYTRULO, monitor for clinical signs and symptoms of infections. If serious infection occurs, administer appropriate treatment and consider withholding VYVGART HYTRULO until the infection has resolved.

### Immunization

Immunization with vaccines during VYVGART HYTRULO treatment has not been studied. The safety of immunization with live or live-attenuated vaccines and the response to immunization with any vaccine are unknown. Because VYVGART HYTRULO causes a reduction in IgG levels, vaccination with live-attenuated or live vaccines is not recommended during treatment with VYVGART HYTRULO. Evaluate the need to administer age-appropriate vaccines according to immunization guidelines before initiation of a new treatment cycle with VYVGART HYTRULO.

### 5.2 Hypersensitivity Reactions

Hypersensitivity reactions, including rash, angioedema, and dyspnea were observed in patients treated with VYVGART HYTRULO and efgartigimod alfa-fcab. Urticaria was also observed in patients treated with VYVGART HYTRULO. In clinical trials, hypersensitivity reactions were mild or moderate, occurred within one hour to three weeks of administration, and did not lead to treatment discontinuation. Healthcare professionals should monitor for clinical signs and symptoms of hypersensitivity reactions for at least 30 minutes after administration [see Dosage and Administration (2.4)]. If a hypersensitivity reaction occurs, the healthcare professional should institute appropriate measures if needed or the patient should seek medical attention.

### 6 Adverse Reactions

The following clinically significant adverse reactions are described elsewhere in the labeling:
Infections [see Warnings and Precautions (5.1)]
Hypersensitivity Reactions [see Warnings and Precautions (5.2)]

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

The safety of efgartigimod alfa in patients with gMG was established in a double blinded placebo-controlled study with efgartigimod alfa-fcab administered intravenously (Study 1) and its open-label extension, and in an active-controlled study of VYVGART HYTRULO administered subcutaneously (Study 2) and its open-label extension.

Adverse Reactions with Efgartigimod Alfa-fcab Intravenous

In clinical studies, the safety of efgartigimod alfa-fcab administered intravenously has been evaluated in 246 patients who received at least one dose of efgartigimod alfa-fcab, including 57 patients exposed to at least 7 treatment cycles and 8 patients exposed to at least 10 treatment cycles.

In a placebo-controlled study (Study 1) in patients with gMG, 84 patients received efgartigimod alfa-fcab 10 mg/kg [see Clinical Studies (14)]. Of these 84 patients, approximately 75% were female, 82% were White, 11% were Asian, and 8% were of Hispanic or Latino ethnicity. The mean age at study entry was 46 years (range 19 to 78).

The minimum time to initiate a subsequent cycle, specified by study protocol, was 50 days from the start of the previous treatment cycle. On average, efgartigimod alfa-fcab-treated patients received 2 cycles in Study 1. The mean and median times to the second treatment cycle were 94 days and 72 days from the initial infusion of the first treatment cycle, respectively, for efgartigimod alfa-fcab-treated patients.

Adverse reactions reported in at least 5% of patients treated with efgartigimod alfa-fcab and more frequently than placebo are summarized in Table 4. The most common adverse reactions (reported in at least 10% of efgartigimod alfa-fcab-treated patients) were respiratory tract infection, headache, and urinary tract infection.

TABLE 4
Adverse Reactions in at least 5% of Patients Treated with Efgartigimod
Alfa-fcab Intravenously (EFG IV) and More Frequently than in
Placebo-Treated Patients in Study 1 (Safety Population)

| | EFG IV | Placebo |
|---|---|---|
| Adverse | (N = 84) | (N = 83) |
| reaction | % | % |
| Respiratory tract infection | 33 | 29 |
| Headache* | 32 | 29 |
| Urinary tract infection | 10 | 5 |
| Paraesthesia† | 7 | 5 |
| Myalgia | 6 | 1 |

*Headache includes migraine and procedural headache.
†Paraesthesia includes oral hypoesthesia, hypoesthesia, and hyperesthesia.

Adverse Reactions with VYVGART HYTRULO

In an active-controlled study in patients with gMG (Study 2), 110 patients were randomized and received one cycle of once weekly administrations for 4 weeks (4 administrations total), of either VYVGART HYTRULO subcutaneously (n=55) or efgartigimod alfa-fcab intravenously (n=55) at recommended doses [see Dosage and Administration (2.2)]. The open-label extension of Study 2 included some patients who switched from efgartigimod alfa-fcab IV to VYVGART HYTRULO.

The most common adverse reactions (reported in at least 10% of VYVGART HYTRULO-treated patients) were injection site reactions and headache.

In Study 2, injection site reactions occurred in 38% of patients receiving VYVGART HYTRULO. These were injection site rash, erythema, pruritus, bruising, pain, and urticaria.

In the Study 2 and its open-label extension (n=168), all injection site reactions were mild to moderate in severity and did not lead to treatment discontinuation. The majority occurred within 24 hours after administration and resolved spontaneously. Most injection site reactions occurred during the first treatment cycle, and the incidence decreased with each subsequent cycle.

### 7 Drug Interactions

### 7.1 Effect of VYVGART HYTRULO on Other Drugs

Concomitant use of VYVGART HYTRULO with medications that bind to the human neonatal Fc receptor (FcRn) (e.g., immunoglobulin products, monoclonal antibodies, or antibody derivates containing the human Fc domain of the IgG subclass) may lower systemic exposures and reduce effectiveness of such medications. Closely monitor for reduced effectiveness of medications that bind to the human neonatal Fc receptor. When concomitant long-term use of such medications is essential for patient care, consider discontinuing VYVGART HYTRULO and using alternative therapies.

### 8 Use in Specific Populations

### 8.1 Pregnancy

### Risk Summary

There are no available data on the use of VYVGART HYTRULO or efgartigimod alfa containing products during pregnancy. There was no evidence of adverse developmental outcomes following the intravenous administration of efgartigimod alfa at up to 100 mg/kg/day in rats and rabbits (see Data).

The background rate of major birth defects and miscarriage in the indicated population is unknown. In the U.S. general population, the estimated background rate of major birth defects and miscarriage in clinically recognized pregnancies is 2% to 4% and 15% to 20%, respectively.

### Clinical Considerations

### Fetal/Neonatal Adverse Reactions

Monoclonal antibodies are increasingly transported across the placenta as pregnancy progresses, with the largest amount transferred during the third trimester. Therefore, efgartigimod alfa may be transmitted from the mother to the developing fetus.

As VYVGART HYTRULO is expected to reduce maternal IgG antibody levels, reduction in passive protection to the newborn is anticipated. Risk and benefits should be considered prior to administering live or live-attenuated vaccines to infants exposed to VYVGART HYTRULO in utero [see Warnings and Precautions (5.1)].

### Data

### Animal Data

VYVGART HYTRULO for subcutaneous injection contains efgartigimod alfa and hyaluronidase [see Description (11)].

### Efgartigimod Alfa:

Intravenous administration of efgartigimod alfa (0, 30, or 100 mg/kg/day) to pregnant rats and rabbits throughout organogenesis resulted in no adverse effects on embryofetal development in either species. Maternal efgartigimod alfa exposures at the highest no-effect doses were approximately 8 and 62 times, respectively, that in humans at the recommended human dose (RHD) of 1008 mg.
Intravenous administration of efgartigimod alfa (0, 30, or 100 mg/kg/day) to rats throughout gestation and lactation resulted in no adverse effects on pre- or postnatal development. Maternal exposures at the highest no-effect dose were approximately 13 times that in humans at the RHD.

### Hyaluronidase:

In a study in which hyaluronidase (recombinant human) was administered by subcutaneous injection to pregnant mice throughout organogenesis, increased embryofetal mortality and decreased fetal body weights were observed at the highest doses tested. The no-effect dose for adverse effects on embryofetal development in the mouse was approximately 1800 times the dose of hyaluronidase at the recommended human dose (RHD) of VYVGART HYTRULO (1,008 mg efgartigimod alfa and 11,200 U hyaluronidase), on a U/kg basis.
There were no adverse effects on pre- and postnatal development following subcutaneous administration of hyaluronidase (recombinant human) to mice throughout gestation and lactation at doses up to 5,000 times the dose of hyaluronidase at the RHD of VYVGART HYTRULO, on a U/kg basis.

### 8.2 Lactation

### Risk Summary

There is no information regarding the presence of efgartigimod alfa or hyaluronidase, from administration of VYVGART HYTRULO, in human milk, the effects on the breastfed infant, or the effects on milk production. Maternal IgG is known to be present in human milk.

The developmental and health benefits of breastfeeding should be considered along with the mother's clinical need for VYVGART HYTRULO and any potential adverse effects on the breastfed infant from VYVGART HYTRULO or from the underlying maternal condition.

### 8.4 Pediatric Use

Safety and effectiveness in pediatric patients have not been established.

### 8.5 Geriatric Use

Clinical studies of VYVGART HYTRULO did not include sufficient numbers of patients aged 65 and older to determine whether they respond differently from younger adult patients.

### 8.6 Renal Impairment

No dose adjustment of VYVGART HYTRULO is needed for patients with mild renal impairment. There are insufficient data to evaluate the impact of moderate renal impairment (eGFR 30-59 mL/min/1.73 m²) and severe renal impairment (eGFR <30 mL/min/1.73 m²) on pharmacokinetic parameters of VYVGART HYTRULO [see Clinical Pharmacology (12.3)].

### 11 DESCRIPTION

VYVGART HYTRULO is a coformulation of efgartigimod alfa and hyaluronidase (human recombinant).

Efgartigimod alfa, a neonatal Fc receptor blocker, is a human immunoglobulin G1 (IgG1)-derived Fc fragment (fragment, crystallized) of the za allotype, produced in Chinese hamster ovary (CHO) cells. The efgartigimod alfa Fc fragment is a homodimer consisting of two identical peptide chains each consisting of 227 amino acids linked together by two interchain disulfide bonds with affinity for FcRn. The molecular weight of efgartigimod alfa is approximately 54 kDa.

Hyaluronidase (human recombinant) is an endoglycosidase used to increase the dispersion and absorption of co-administered drugs when administered subcutaneously. Hyaluronidase (human recombinant) is a glycosylated single-chain protein produced by Chinese hamster ovary cells containing a DNA plasmid encoding for a soluble fragment of human hyaluronidase (PH20). Hyaluronidase (human recombinant) has a molecular weight of approximately 61 kDa.

VYVGART HYTRULO (efgartigimod alfa and hyaluronidase-qvfc) injection is a sterile, preservative free, yellowish, clear to opalescent solution supplied in a single-dose vial for subcutaneous injection.

Each 5.6 mL single-dose vial of VYVGART HYTRULO contains 1,008 mg efgartigimod alfa and 11,200 units hyaluronidase (human recombinant). Each mL of solution contains 180 mg of efgartigimod alfa, 2,000 units of hyaluronidase (human recombinant) and histidine (1.4 mg), L-histidine hydrochloride monohydrate (2.2 mg), methionine (1.5 mg), polysorbate 20 (0.4 mg), sodium chloride (5.8 mg), sucrose (20.5 mg), and water for injection, USP, at a pH of 6.0.

### 12 Clinical Pharmacology

### 12.1 Mechanism of Action

VYVGART HYTRULO is a coformulation of efgartigimod alfa and hyaluronidase.

Efgartigimod alfa is a human IgG1 antibody fragment that binds to the neonatal Fc receptor (FcRn), resulting in the reduction of circulating IgG.

Hyaluronidase increases permeability of the subcutaneous tissue by depolymerizing hyaluronan. This effect is transient and permeability of the subcutaneous tissue is restored within 24 to 48 hours.

### 12.2 Pharmacodynamics

In Study 1 [see Clinical Studies (14)], the pharmacological effect of efgartigimod alfa-fcab was assessed by measuring the decrease in serum IgG levels and AChR autoantibody levels. In patients testing positive for AChR antibodies and who were treated with efgartigimod alfa-fcab intravenous, there was a reduction in total IgG levels relative to baseline. Decrease in AChR autoantibody levels followed a similar pattern. A decrease in AChR-Ab was associated with a clinical response in AChR-Ab positive patients, as measured by the change from baseline in MG-ADL total score.

In Study 2, the pharmacological effect of VYVGART HYTRULO administered subcutaneously (SC) at 1,008 mg/11,200 Units was compared to efgartigimod alfa-fcab administered intravenously at 10 mg/kg (EFG IV) in gMG patients. The maximum mean reduction in AChR-Ab level was observed at week 4, with a mean reduction of 62.2% and 59.7% in the VYVGART HYTRULO SC and efgartigimod alfa-fcab IV arm, respectively. The decrease in total IgG levels followed a similar pattern. The 90% confidence intervals for the geometric mean ratios of AChR-Ab reduction at day 29 and AUEC_{0-4w} (area under the effect-time curve from time 0 to 4 weeks post dose) were within the range of 80% to 125%, indicating no clinically significant difference between the two formulations.

### 12.3 Pharmacokinetics

Efgartigimod alfa exposures were approximately dose-proportional up to the highest subcutaneously tested dose of VYVGART HYTRULO (1750 mg, 1.75 times the recommended dosage).

### Distribution

The volume of distribution is 15 to 20 L.

### Metabolism and Elimination

Efgartigimod alfa and hyaluronidase are expected to be degraded by proteolytic enzymes into small peptides and amino acids.

The terminal half-life is 80 to 120 hours (3 to 5 days).

After a single intravenous dose of 10 mg/kg efgartigimod alfa-fcab in healthy subjects, less than 0.1% of the administered dose was recovered in urine.

### Specific Populations

### Age, Sex and Race

A population pharmacokinetics analysis assessing the effects of age, body weight, sex, and race did not suggest any clinically significant impact of these covariates on efgartigimod alfa exposures.

### Body Weight

A population pharmacokinetics analysis suggests that the influence of body weight on efgartigimod alfa exposure after administration of VYVGART HYTRULO SC 1008 mg was limited and not clinically relevant.

Patients with Renal Impairment

No dedicated pharmacokinetic study has been performed in patients with renal impairment.

A population PK analysis of data from the VYVGART HYTRULO clinical studies indicated that patients with mild renal impairment (eGFR 60-89 mL/min/1.73 m2) had 11% increase in exposure relative to the exposure in patients with normal renal function [see Use in Specific Populations (8.6)].

Patients with Hepatic Impairment

No dedicated pharmacokinetic study has been performed in patients with hepatic impairment. Hepatic impairment is not expected to affect the pharmacokinetics of efgartigimod alfa.

### Drug Interaction Studies

Clinical drug interactions studies have not been performed with efgartigimod alfa.

### P450 Enzymes

Efgartigimod alfa is not metabolized by cytochrome P450 enzymes; therefore, interactions with concomitant medications that are substrates, inducers, or inhibitors of cytochrome P450 enzymes are unlikely.

Drug Interactions with Other Drugs or Biological Products

Efgartigimod alfa may decrease concentrations of compounds that bind to the human FcRn [see Drug Interactions (7.1)].

### 12.6 Immunogenicity

The observed incidence of anti-drug antibodies is highly dependent on the sensitivity and specificity of the assay. Differences in assay methods preclude meaningful comparisons of the incidence of anti-drug antibodies in the studies described below with the incidence of anti-drug antibodies in other studies, including those of VYVGART HYTRULO or of other efgartigimod products.

In up to 10 weeks treatment in Study 2, the incidence of anti-efgartigimod alfa antibodies was 35% (19/55) following treatment with VYVGART HYTRULO and 20% (11/55) in patients receiving intravenous efgartigimod alfa-fcab. For both IV and SC arms, neutralizing anti-efgartigimod alfa antibodies were detected in 4% (2/55) of patients. Some neutralizing antibodies may not be detected by the assay. The available data are too limited to make definitive conclusions regarding immunogenicity and the effect on pharmacokinetics, safety, or efficacy of VYVGART HYTRULO.

### 13 Nonclinical Toxicology

### 13.1 Carcinogenesis, Mutagenesis, Impairment of Fertility

VYVGART HYTRULO for subcutaneous injection contains efgartigimod alfa and hyaluronidase [see Description (11)].

### Carcinogenesis and Mutagenesis

No studies have been conducted to assess the carcinogenic potential of efgartigimod alfa.

No studies have been conducted to assess the genotoxic potential of efgartigimod alfa.

No carcinogenicity or genotoxicity studies were conducted for human recombinant hyaluronidase.

### Impairment of Fertility

Intravenous administration of efgartigimod alfa (0, 30, or 100 mg/kg/day) to male and female rats prior to and during mating and continuing in females through gestation day 7 resulted in no adverse effects on fertility. Efgartigimod alfa exposures at the highest no-effect dose were approximately 12 times that in humans at the recommended human dose of 1008 mg.

There were no effects on reproductive tissues in monkeys following subcutaneous administration of hyaluronidase (recombinant human) doses up to approximately 1,200 times the dose of hyaluronidase at the recommended human dose (RHD) of VYVGART HYTRULO (1,008 mg efgartigimod alfa and 11,200 U hyaluronidase) on a U/kg basis for 39 weeks. No systemic exposure to hyaluronidase was observed at doses up to approximately 120 times the dose of hyaluronidase at the RHD of VYVGART HYTRULO, on a U/kg basis.

### 14 Clinical Studies

Study 1 (described below) which established the effectiveness of efgartigimod alfa-fcab for the treatment of generalized myasthenia gravis (gMG) in adults who are AChR antibody positive was conducted with efgartigimod alfa-fcab intravenous formulation. In Study 2, VYVGART HYTRULO demonstrated a comparable pharmacodynamic effect on AChR antibody reduction as compared to the efgartigimod alfa-fcab intravenous formulation, which established the efficacy of VYVGART HYTRULO [see Clinical Pharmacology (12.2)].

### Study 1 (Efgartigimod Alfa-Fcab Intravenous)

The efficacy of efgartigimod alfa-fcab intravenous (EFG IV) for the treatment of generalized myasthenia gravis (gMG) in adults who are AChR antibody positive was established in a 26-week, multicenter, randomized, double-blind, placebo-controlled trial (Study 1; NCT03669588).

Study 1 enrolled patients who met the following criteria at screening:
Myasthenia Gravis Foundation of America (MGFA) clinical classification class II to IV
MG-Activities of Daily Living (MG-ADL) total score of ≥5.
On stable dose of MG therapy prior to screening, that included acetylcholinesterase (AChE) inhibitors, steroids, or non-steroidal immunosuppressive therapies (NSISTs), either in combination or alone
IgG levels of at least 6 g/L

A total of 167 patients were enrolled in Study 1 and were randomized to receive either EFG IV 10 mg/kg (1200 mg for those weighing 120 kg or more) (n=84) or placebo (n=83). Baseline characteristics were similar between treatment groups. Patients had a median age of 46 years at screening (range: 19 to 81 years) and a median time since diagnosis of 9 years. Seventy-one percent were female, and 84% were White. Median MG-ADL total score was 9, and median Quantitative Myasthenia Gravis (QMG) total score was 16. The majority of patients (n=65 for EFG IV; n=64 for placebo) were positive for AChR antibodies.

At baseline, over 80% of patients in each group received AChE inhibitors, over 70% in each treatment group received steroids, and approximately 60% in each treatment group received NSISTs, at stable doses.

Patients were treated with 10 mg/kg EFG IV administered as an intravenous infusion over one hour once weekly for 4 weeks. In patients weighing 120 kg or more, EFG IV was administered as 1200 mg per infusion. Subsequent treatment cycles were administered based on clinical evaluation, but no sooner than 50 days from the start of the previous treatment cycle.

The efficacy of EFG IV was measured using the Myasthenia Gravis-Specific Activities of Daily Living scale (MG-ADL) which assesses the impact of gMG on daily functions of 8 signs or symptoms that are typically affected in gMG. Each item is assessed on a 4-point scale where a score of 0 represents normal function and a score of 3 represents loss of ability to perform that function. A total score ranges from 0 to 24, with the higher scores indicating more impairment. In this study, an MG-ADL responder was defined as a patient with a 2-point or greater reduction in the total MG-ADL score compared to the treatment cycle baseline for at least 4 consecutive weeks, with the first reduction occurring no later than 1 week after the last infusion of the cycle.

The primary efficacy endpoint was the comparison of the percentage of MG-ADL responders during the first treatment cycle between treatment groups in the AChR-Ab positive population. A statistically significant difference favoring EFG IV was observed in the MG-ADL responder rate during the first treatment cycle [67.7% in the EFG IV-treated group vs 29.7% in the placebo-treated group (p<0.0001)].

The efficacy of EFG IV was also measured using the Quantitative Myasthenia Gravis (QMG) total score which is a 13-item categorical grading system that assesses muscle weakness. Each item is assessed on a 4-point scale where a score of 0 represents no weakness and a score of 3 represents severe weakness. A total possible score ranges from 0 to 39, where higher scores indicate more severe impairment. In this study, a QMG responder was defined as a patient who had a 3-point or greater reduction in the total QMG score compared to the treatment cycle baseline for at least 4 consecutive weeks, with the first reduction occurring no later than 1 week after last infusion of the cycle.

The secondary endpoint was the comparison of the percentage of QMG responders during the first treatment cycle between both treatment groups in the AChR-Ab positive patients. A statistically significant difference favoring EFG IV was observed in the QMG responder rate during the first treatment cycle [63.1% in the EFG IV-treated group vs 14.1% in the placebo-treated group (p<0.0001)].

The results are presented in Table 5.

TABLE 5
MG-ADL and QMG Responders During Cycle 1 in
AChR-Ab Positive Patients (mITT Analysis Set)

| | EFG IV | Placebo | | Odds | |
|---|---|---|---|---|---|
| | n = 65 | n = 64 | | Ratio | |
| | % | % | P-value | (95% CI) | |
| MG-ADL | 67.7 | 29.7 | <0.0001 | 4.951 | (2.213, 11.528) |
| Responders | | | | | |
| QMG | 63.1 | 14.1 | <0.0001 | 10.842 | (4.179, 31.200) |
| Responders | | | | | |

EFG IV = Efgartigimod alfa-fcab intravenous;
MG-ADL = Myasthenia Gravis Activities of Daily Living;
QMG = Quantitative Myasthenia Gravis;
mITT = modified intent-to-treat;
n = number of patients for whom the observation was reported;
CI = confidence interval;
Logistic regression stratified for AChR-Ab status (if applicable), Japanese/Non-Japanese and standard of care, with baseline MG-ADL as covariate/QMG as covariates
Two-sided exact p-value

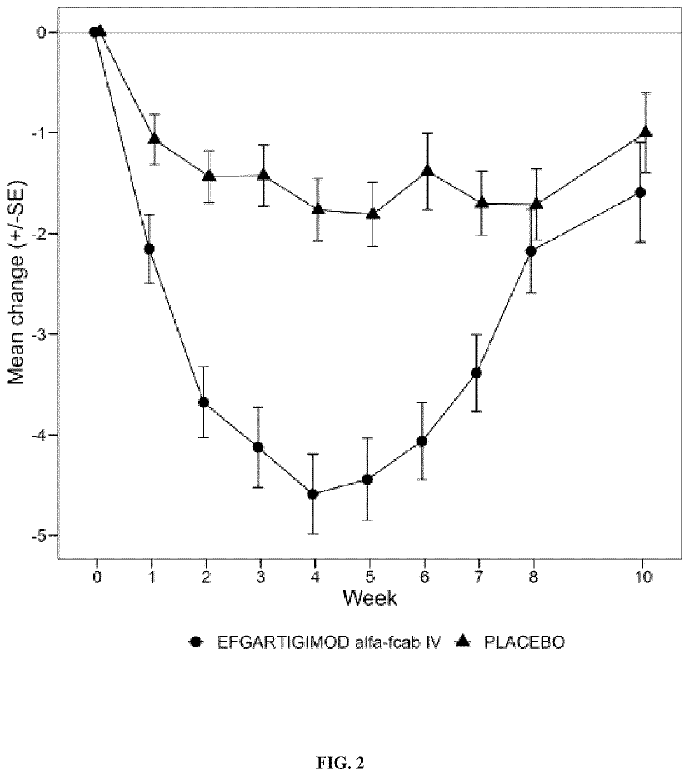

FIG. 2 shows the mean change from baseline on the MG-ADL during cycle 1.

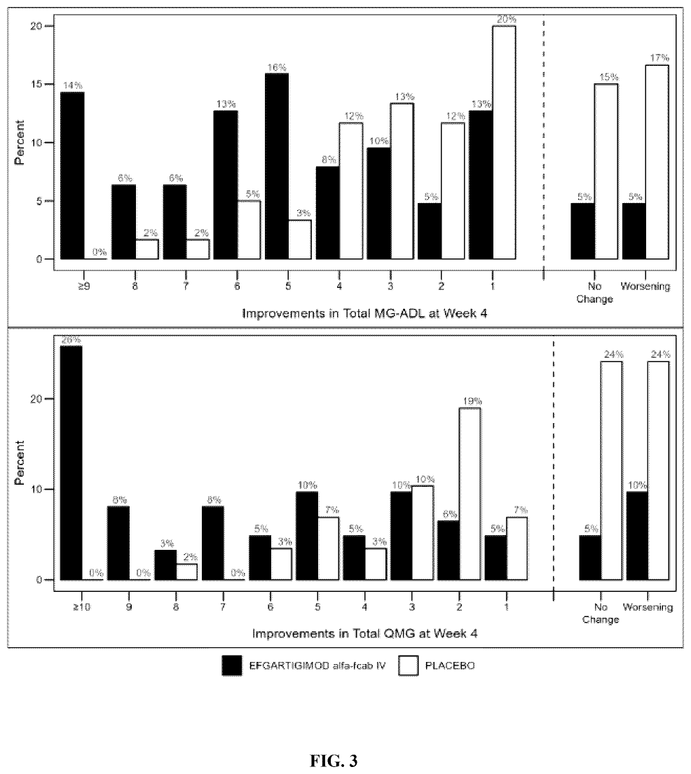

FIG. 3 shows the distribution of response on the MG-ADL and QMG during cycle 1, four weeks after the first infusion of EFG IV.

### 16 how Supplied/Storage and Handling

VYVGART HYTRULO (efgartigimod alfa and hyaluronidase-qvfc) injection is a preservative free, sterile, yellowish, clear to opalescent solution supplied as one single-dose vial per carton containing 1,008 mg efgartigimod alfa and 11,200 units hyaluronidase per 5.6 mL (180 mg/2,000 units per mL):(NDC 73475-3102-3).

Store VYVGART HYTRULO vials refrigerated at 2° C. to 8° C. (36° F. to 46° F.) in the original carton to protect from light until time of use. Do not freeze. Do not shake.

If needed, unopened vials may be stored in the original carton for up to 3 days at room temperature at 20° C. to 25° C. (68° F. to 77° F.) for a single period before administration or returned to refrigeration. Do not store the vial at room temperature more than one time. Record the date removed from and the date returned to the refrigerator on the carton.

### 17 Patient Counseling Information

### Infections

Instruct patients to communicate any history of infections to the healthcare provider and to contact their healthcare provider if they develop any symptoms of an infection. Advise patients to complete age-appropriate vaccines according to immunization guidelines prior to initiation of a new treatment cycle with VYVGART HYTRULO. Administration of live or live-attenuated vaccines is not recommended during treatment with VYVGART HYTRULO [see Warnings and Precautions (5.1)].

### Hypersensitivity Reactions

Inform patients about the signs and symptoms of hypersensitivity reactions. Advise patients to contact their healthcare provider immediately for signs or symptoms of hypersensitivity reactions [see Warnings and Precautions (5.2)].

### Example 2: Updated Label with Prescribing Information

The text below is from the revised label with the prescribing information for VYVGART® HYTRULO (efgartigimod alfa and hyaluronidase-qvfc) injection, for subcutaneous use, as approved by Food and Drug Administration under BLA 761304 (Initial U.S. Approval: June 2023; Revised January 2024). Sections from the label that are identical to the label included in Example 1 have been omitted.

### Highlights of Prescribing Information

These highlights do not include all the information needed to use VYVGART HYTRULO safely and effectively. See full prescribing information for VYVGART HYTRULO.

VYVGART® HYTRULO (efgartigimod alfa and hyaluronidase-qvfc) injection, for subcutaneous use Initial U.S. Approval: 2023

### Recent Major Changes

### Contraindications (4) December 2023

Warnings and Precautions (5.2, 5.3) December 2023

### Indications and Usage

VYVGART HYTRULO is a combination of efgartigimod alfa, a neonatal Fc receptor blocker, and hyaluronidase, an endoglycosidase, indicated for the treatment of generalized myasthenia gravis (gMG) in adult patients who are anti-acetylcholine receptor (AChR) antibody positive. (1)

### Dosage and Administration

See Full Prescribing Information for instructions on dosage, preparation, and administration. (2.1, 2.2, 2.3, 2.4)

Evaluate the need to administer age-appropriate vaccines according to immunization guidelines before initiation of a new treatment cycle with VYVGART HYTRULO. (2.1)

Administer by a healthcare professional only. (2.2)

For subcutaneous use with a winged infusion set. (2.2)

The recommended dose is 1,008 mg/11,200 units (1,008 mg efgartigimod alfa and 11,200 units hyaluronidase) administered subcutaneously over approximately 30 to 90 seconds in cycles of once weekly injections for 4 weeks. (2.3)

Administer subsequent treatment cycles based on clinical evaluation; safety of initiating subsequent cycles sooner than 50 days from the start of the previous treatment cycle has not been established. (2.3)

### Dosage Forms and Strengths

Injection: 1,008 mg efgartigimod alfa and 11,200 units hyaluronidase per 5.6 mL (180 mg/2,000 units per mL) in a single-dose vial. (3)

### Contraindications

VYVGART HYTRULO is contraindicated in patients with serious hypersensitivity to efgartigimod alfa products, to hyaluronidase, or to any of the excipients of VYVGART HYTRULO. (4)

### Warnings and Precautions

Infections: Delay administration of VYVGART HYTRULO to patients with an active infection. Monitor for signs and symptoms of infection in patients treated with VYVGART HYTRULO. If serious infection occurs, administer appropriate treatment and consider withholding VYVGART HYTRULO until the infection has resolved. (5.1)

Hypersensitivity Reactions: Anaphylaxis, hypotension leading to syncope, angioedema, dyspnea, rash, and urticaria have occurred in patients treated with VYVGART HYTRULO or intravenous efgartigimod alfa-fcab product. If a hypersensitivity reaction occurs, the healthcare professional should institute appropriate measures if needed or the patient should seek medical attention. (4, 5.2)

Infusion-Related Reactions: If a severe infusion-related reaction occurs, initiate appropriate therapy; consider the risks and benefits of readministering. If a mild to moderate infusion-related reaction occurs, may rechallenge with close clinical observation, slower infusion rates, and pre-medications. (5.3)

### Adverse Reactions

The most common adverse reactions (≥10%) in patients with gMG treated with efgartigimod alfa-fcab were respiratory tract infections, headache, and urinary tract infection.

Additional common adverse reactions with VYVGART HYTRULO are injection site reactions. (6.1)

To report SUSPECTED ADVERSE REACTIONS, contact Argenx at 1-833-argx411 or FDA at 1-800-FDA-1088 or fda.gov/medwatch.

### Drug Interactions

Closely monitor for reduced effectiveness of medications that bind to the human neonatal Fc receptor. When concomitant long-term use of such medications is essential for patient care, consider discontinuing VYVGART HYTRULO and using alternative therapies. (7)

See 17 for PATIENT COUNSELING INFORMATION.

### Full Prescribing Information: Contents*

1 INDICATIONS AND USAGE
2 DOSAGE AND ADMINISTRATION
2.1 Recommended Vaccination
2.2 Important Dosage and Administration Instructions
2.3 Recommended Dose and Dose Schedules
2.4 Preparation and Administration Instructions
3 DOSAGE FORMS AND STRENGTHS
4 CONTRAINDICATIONS
5 WARNINGS AND PRECAUTIONS
5.1 Infections
5.2 Hypersensitivity Reactions
5.3 Infusion-Related Reactions
6 ADVERSE REACTIONS
6.1 Clinical Trials Experience
6.2 Postmarketing Experience
7 DRUG INTERACTIONS
7.1 Effect of VYVGART HYTRULO on Other Drugs
8 USE IN SPECIFIC POPULATIONS
8.1 Pregnancy
8.2 Lactation
8.4 Pediatric Use
8.5 Geriatric Use
8.6 Renal Impairment
11 DESCRIPTION
12 CLINICAL PHARMACOLOGY
12.1 Mechanism of Action
12.2 Pharmacodynamics
12.3 Pharmacokinetics
12.6 Immunogenicity
13 NONCLINICAL TOXICOLOGY
13.1 Carcinogenesis, Mutagenesis, Impairment of Fertility
14 CLINICAL STUDIES
16 HOW SUPPLIED/STORAGE AND HANDLING
17 PATIENT COUNSELING INFORMATION
Sections or subsections omitted from the full prescribing information are not listed.

### 4 Contraindications

VYVGART HYTRULO is contraindicated in patients with serious hypersensitivity to efgartigimod alfa products, to hyaluronidase, or to any of the excipients of VYVGART HYTRULO. Reactions have included anaphylaxis and hypotension leading to syncope [see Warnings and Precautions (5.2)].

### 5 Warnings and Precautions

### 5.1 Infections

VYVGART HYTRULO may increase the risk of infection. The most common infections observed in Study 1 were urinary tract infection (10% of efgartigimod alfa-fcab-treated patients compared to 5% of placebo-treated patients) and respiratory tract infections (33% of efgartigimod alfa-fcab-treated patients compared to 29% of placebo-treated patients) [see Adverse Reactions (6.1) and Clinical Studies (14)]. A higher frequency of patients who received efgartigimod alfa-fcab compared to placebo were observed to have below normal levels for white blood cell counts (12% versus 5%, respectively), lymphocyte counts (28% versus 19%, respectively), and neutrophil counts (13% versus 6%, respectively). The majority of infections and hematologic abnormalities were mild to moderate in severity. Delay VYVGART HYTRULO administration in patients with an active infection until the infection is resolved. During treatment with VYVGART HYTRULO, monitor for clinical signs and symptoms of infections. If serious infection occurs, administer appropriate treatment and consider withholding VYVGART HYTRULO until the infection has resolved.

### Immunization

Immunization with vaccines during VYVGART HYTRULO treatment has not been studied. The safety of immunization with live or live-attenuated vaccines and the response to immunization with any vaccine are unknown. Because VYVGART HYTRULO causes a reduction in IgG levels, vaccination with live-attenuated or live vaccines is not recommended during treatment with VYVGART HYTRULO. Evaluate the need to administer age-appropriate vaccines according to immunization guidelines before initiation of a new treatment cycle with VYVGART HYTRULO.

### 5.2 Hypersensitivity Reactions

In clinical trials, hypersensitivity reactions, including rash, angioedema, and dyspnea were observed in patients treated with VYVGART HYTRULO or intravenous efgartigimod alfa-fcab. Urticaria was also observed in patients treated with VYVGART HYTRULO. Hypersensitivity reactions were mild or moderate, occurred within one hour to three weeks of administration, and did not lead to treatment discontinuation.

Anaphylaxis and hypotension leading to syncope have been reported in postmarketing experience with intravenous efgartigimod alfa-fcab. Anaphylaxis and hypotension occurred during or within an hour of administration and led to infusion discontinuation and in some cases to permanent treatment discontinuation.

Healthcare professionals should monitor for clinical signs and symptoms of hypersensitivity reactions for at least 30 minutes after administration [see Dosage and Administration (2.4)]. If a hypersensitivity reaction occurs, the healthcare professional should institute appropriate measures if needed or the patient should seek medical attention. VYVGART HYTRULO is contraindicated in patients with a history of serious hypersensitivity to efgartigimod alfa products, to hyaluronidase, or to any of the excipients of VYVGART HYTRULO [see Contraindications (4)].

### 5.3 Infusion-Related Reactions

Infusion-related reactions have been reported with intravenous efgartigimod alfa-fcab in postmarketing experience. The most frequent symptoms and signs were hypertension, chills, shivering, and thoracic, abdominal, and back pain. Infusion-related reactions occurred during or within an hour of administration and led to infusion discontinuation. If a severe infusion-related reaction occurs, initiate appropriate therapy. Consider the risks and benefits of readministering VYVGART HYTRULO following a severe infusion-related reaction. If a mild to moderate infusion-related reaction occurs, patients may be rechallenged with close clinical observation, slower infusion rates, and pre-medications.

### 6 Adverse Reactions

The following clinically significant adverse reactions are described elsewhere in the labeling:
Infections [see Warnings and Precautions (5.1)]
Hypersensitivity Reactions [see Warnings and Precautions (5.2)]
Infusion-Related Reactions [see Warnings and Precautions (5.3)]

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

The safety of efgartigimod alfa in patients with gMG was established in a double blinded placebo-controlled study with efgartigimod alfa-fcab administered intravenously (Study 1) and its open-label extension, and in an active-controlled study of VYVGART HYTRULO administered subcutaneously (Study 2) and its open-label extension.

Adverse Reactions with Efgartigimod Alfa-fcab Intravenous

In clinical studies, the safety of efgartigimod alfa-fcab administered intravenously has been evaluated in 246 patients who received at least one dose of efgartigimod alfa-fcab, including 57 patients exposed to at least 7 treatment cycles and 8 patients exposed to at least 10 treatment cycles.

In a placebo-controlled study (Study 1) in patients with gMG, 84 patients received efgartigimod alfa-fcab 10 mg/kg [see Clinical Studies (14)]. Of these 84 patients, approximately 75% were female, 82% were White, 11% were Asian, and 8% were of Hispanic or Latino ethnicity. The mean age at study entry was 46 years (range 19 to 78).

The minimum time to initiate a subsequent cycle, specified by study protocol, was 50 days from the start of the previous treatment cycle. On average, efgartigimod alfa-fcab-treated patients received 2 cycles in Study 1. The mean and median times to the second treatment cycle were 94 days and 72 days from the initial infusion of the first treatment cycle, respectively, for efgartigimod alfa-fcab-treated patients.

Adverse reactions reported in at least 5% of patients treated with efgartigimod alfa-fcab and more frequently than placebo are summarized in Table 4. The most common adverse reactions (reported in at least 10% of efgartigimod alfa-fcab-treated patients) were respiratory tract infection, headache, and urinary tract infection.

Adverse Reactions with VYVGART HYTRULO

In an active-controlled study in patients with gMG (Study 2), 110 patients were randomized and received one cycle of once weekly administrations for 4 weeks (4 administrations total), of either VYVGART HYTRULO subcutaneously (n=55) or efgartigimod alfa-fcab intravenously (n=55) at recommended doses [see Dosage and Administration (2.2)]. The open-label extension of Study 2 included some patients who switched from efgartigimod alfa-fcab IV to VYVGART HYTRULO.

The most common adverse reactions (reported in at least 10% of VYVGART HYTRULO-treated patients) were injection site reactions and headache.

In Study 2, injection site reactions occurred in 38% of patients receiving VYVGART HYTRULO. These were injection site rash, erythema, pruritus, bruising, pain, and urticaria.

In the Study 2 and its open-label extension (n=168), all injection site reactions were mild to moderate in severity and did not lead to treatment discontinuation. The majority occurred within 24 hours after administration and resolved spontaneously. Most injection site reactions occurred during the first treatment cycle, and the incidence decreased with each subsequent cycle.

### 6.2 Postmarketing Experience

The following adverse reactions have been identified during postapproval use of efgartigimod alfa products. Because these reactions are reported voluntarily from a population of uncertain size, it is not always possible to reliably estimate their frequency or establish a causal relationship to drug exposure.

Immune System Disorders: Hypersensitivity reactions including anaphylaxis and hypotension, and infusion-related reactions [see Warnings and Precautions (5.2 5.3)].

### 8 Use in Specific Populations

### 8.1 Pregnancy

### Pregnancy Exposure Registry

There is a pregnancy exposure registry that monitors pregnancy outcomes in women exposed to VYVGART HYTRULO during pregnancy. Healthcare providers and patients may call 1-855-272-6524 or go to Vyvgartpregnancy.com to enroll in or to obtain information about the registry.

### Risk Summary

There are no available data on the use of VYVGART HYTRULO or efgartigimod alfa containing products during pregnancy. There was no evidence of adverse developmental outcomes following the intravenous administration of efgartigimod alfa at up to 100 mg/kg/day in rats and rabbits (see Data).

The background rate of major birth defects and miscarriage in the indicated population is unknown. In the U.S. general population, the estimated background rate of major birth defects and miscarriage in clinically recognized pregnancies is 2% to 4% and 15% to 20%, respectively.

### Clinical Considerations

### Fetal/Neonatal Adverse Reactions

Monoclonal antibodies are increasingly transported across the placenta as pregnancy progresses, with the largest amount transferred during the third trimester. Therefore, efgartigimod alfa may be transmitted from the mother to the developing fetus.

As VYVGART HYTRULO is expected to reduce maternal IgG antibody levels, reduction in passive protection to the newborn is anticipated. Risk and benefits should be considered prior to administering live or live-attenuated vaccines to infants exposed to VYVGART HYTRULO in utero [see Warnings and Precautions (5.1)].

### Data

### Animal Data

VYVGART HYTRULO for subcutaneous injection contains efgartigimod alfa and hyaluronidase [see Description (11)].

### Efgartigimod Alfa:

Intravenous administration of efgartigimod alfa (0, 30, or 100 mg/kg/day) to pregnant rats and rabbits throughout organogenesis resulted in no adverse effects on embryofetal development in either species. Maternal efgartigimod alfa exposures at the highest no-effect doses were approximately 8 and 62 times, respectively, that in humans at the recommended human dose (RHD) of 1008 mg.
Intravenous administration of efgartigimod alfa (0, 30, or 100 mg/kg/day) to rats throughout gestation and lactation resulted in no adverse effects on pre- or postnatal development. Maternal exposures at the highest no-effect dose were approximately 13 times that in humans at the RHD.

### Hyaluronidase:

In a study in which hyaluronidase (human recombinant) was administered by subcutaneous injection to pregnant mice throughout organogenesis, increased embryofetal mortality and decreased fetal body weights were observed at the highest doses tested. The no-effect dose for adverse effects on embryofetal development in the mouse was approximately 1800 times the dose of hyaluronidase at the recommended human dose (RHD) of VYVGART HYTRULO (1,008 mg efgartigimod alfa and 11,200 U hyaluronidase), on a U/kg basis.
There were no adverse effects on pre- and postnatal development following subcutaneous administration of hyaluronidase (human recombinant) to mice throughout gestation and lactation at doses up to 5,000 times the dose of hyaluronidase at the RHD of VYVGART HYTRULO, on a U/kg basis.

### 13 Nonclinical Toxicology

### 13.1 Carcinogenesis, Mutagenesis, Impairment of Fertility

VYVGART HYTRULO for subcutaneous injection contains efgartigimod alfa and hyaluronidase [see Description (11)].

### Carcinogenesis and Mutagenesis

No studies have been conducted to assess the carcinogenic potential of efgartigimod alfa.

No studies have been conducted to assess the genotoxic potential of efgartigimod alfa.

No carcinogenicity or genotoxicity studies were conducted for human recombinant hyaluronidase.

### Impairment of Fertility

Intravenous administration of efgartigimod alfa (0, 30, or 100 mg/kg/day) to male and female rats prior to and during mating and continuing in females through gestation day 7 resulted in no adverse effects on fertility. Efgartigimod alfa exposures at the highest no-effect dose were approximately 12 times that in humans at the recommended human dose of 1008 mg.

There were no effects on reproductive tissues in monkeys following subcutaneous administration of hyaluronidase (human recombinant) doses up to approximately 1,200 times the dose of hyaluronidase at the recommended human dose (RHD) of VYVGART HYTRULO (1,008 mg efgartigimod alfa and 11,200 U hyaluronidase) on a U/kg basis for 39 weeks. No systemic exposure to hyaluronidase was observed at doses up to approximately 120 times the dose of hyaluronidase at the RHD of VYVGART HYTRULO, on a U/kg basis.

### 14 Clinical Studies

Study 1 (described below) which established the effectiveness of efgartigimod alfa-fcab for the treatment of generalized myasthenia gravis (gMG) in adults who are AChR antibody positive was conducted with efgartigimod alfa-fcab intravenous formulation. In Study 2, VYVGART HYTRULO demonstrated a comparable pharmacodynamic effect on AChR antibody reduction as compared to the efgartigimod alfa-fcab intravenous formulation, which established the efficacy of VYVGART HYTRULO [see Clinical Pharmacology (12.2)].

### Study 1 (Efgartigimod Alfa-Fcab Intravenous)

The efficacy of efgartigimod alfa-fcab intravenous (EFG IV) for the treatment of generalized myasthenia gravis (gMG) in adults who are AChR antibody positive was established in a 26-week, multicenter, randomized, double-blind, placebo-controlled trial (Study 1; NCT03669588).

Study 1 enrolled patients who met the following criteria at screening:
Myasthenia Gravis Foundation of America (MGFA) clinical classification class II to IV
MG-Activities of Daily Living (MG-ADL) total score of ≥5.
On stable dose of MG therapy prior to screening, that included acetylcholinesterase (AChE) inhibitors, steroids, or non-steroidal immunosuppressive therapies (NSISTs), either in combination or alone
IgG levels of at least 6 g/L

A total of 167 patients were enrolled in Study 1 and were randomized to receive either EFG IV 10 mg/kg (1200 mg for those weighing 120 kg or more) (n=84) or placebo (n=83). Baseline characteristics were similar between treatment groups. Patients had a median age of 46 years at screening (range: 19 to 81 years) and a median time since diagnosis of 7 years. Seventy-one percent were female, and 84% were White. Median MG-ADL total score was 9, and median Quantitative Myasthenia Gravis (QMG) total score was 16. The majority of patients (n=65 for EFG IV; n=64 for placebo) were positive for AChR antibodies.

At baseline, over 80% of patients in each group received AChE inhibitors, over 70% in each treatment group received steroids, and approximately 60% in each treatment group received NSISTs, at stable doses.

Patients were treated with 10 mg/kg EFG IV administered as an intravenous infusion over one hour once weekly for 4 weeks. In patients weighing 120 kg or more, EFG IV was administered as 1200 mg per infusion. Subsequent treatment cycles were administered based on clinical evaluation, but no sooner than 50 days from the start of the previous treatment cycle.

The efficacy of EFG IV was measured using the Myasthenia Gravis-Specific Activities of Daily Living scale (MG-ADL) which assesses the impact of gMG on daily functions of 8 signs or symptoms that are typically affected in gMG. Each item is assessed on a 4-point scale where a score of 0 represents normal function and a score of 3 represents loss of ability to perform that function. A total score ranges from 0 to 24, with the higher scores indicating more impairment. In this study, an MG-ADL responder was defined as a patient with a 2-point or greater reduction in the total MG-ADL score compared to the treatment cycle baseline for at least 4 consecutive weeks, with the first reduction occurring no later than 1 week after the last infusion of the cycle.

The primary efficacy endpoint was the comparison of the percentage of MG-ADL responders during the first treatment cycle between treatment groups in the AChR-Ab positive population. A statistically significant difference favoring EFG IV was observed in the MG-ADL responder rate during the first treatment cycle [67.7% in the EFG IV-treated group vs 29.7% in the placebo-treated group (p<0.0001)].

The efficacy of EFG IV was also measured using the Quantitative Myasthenia Gravis (QMG) total score which is a 13-item categorical grading system that assesses muscle weakness. Each item is assessed on a 4-point scale where a score of 0 represents no weakness and a score of 3 represents severe weakness. A total possible score ranges from 0 to 39, where higher scores indicate more severe impairment. In this study, a QMG responder was defined as a patient who had a 3-point or greater reduction in the total QMG score compared to the treatment cycle baseline for at least 4 consecutive weeks, with the first reduction occurring no later than 1 week after last infusion of the cycle.

The secondary endpoint was the comparison of the percentage of QMG responders during the first treatment cycle between both treatment groups in the AChR-Ab positive patients. A statistically significant difference favoring EFG IV was observed in the QMG responder rate during the first treatment cycle [63.1% in the EFG IV-treated group vs 14.1% in the placebo-treated group (p<0.0001)].

The results are presented in Table 5.

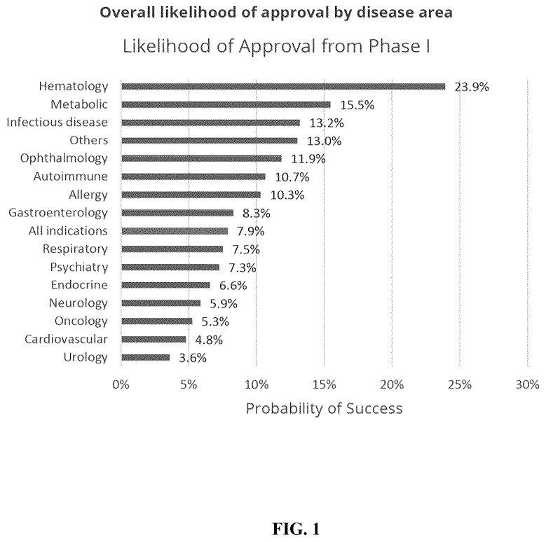

FIG. 1 shows the mean change from baseline on the MG-ADL during cycle 1.

FIG. 2 shows the distribution of response on the MG-ADL and QMG during cycle 1, four weeks after the first infusion of EFG IV.

### 17 Patient Counseling Information

### Pregnancy Registry

There is a pregnancy exposure registry that monitors pregnancy outcomes in women exposed to VYVGART HYTRULO during pregnancy. Encourage participation and advise patients about how they may enroll in the registry [see Use In Specific Populations (8.1)].

### Infections

Instruct patients to communicate any history of infections to the healthcare provider and to contact their healthcare provider if they develop any symptoms of an infection. Advise patients to complete age-appropriate vaccines according to immunization guidelines prior to initiation of a new treatment cycle with VYVGART HYTRULO. Administration of live or live-attenuated vaccines is not recommended during treatment with VYVGART HYTRULO [see Warnings and Precautions (5.1)].

### Hypersensitivity Reactions

Inform patients that hypersensitivity reactions, including angioedema and anaphylaxis, have occurred in patients who were treated with efgartigimod alfa products. Inform patients about the signs and symptoms of these reactions, and advise patients to contact their healthcare provider immediately if these occur [see Warnings and Precautions (5.2)].

### Infusion-Related Reactions

Advise patients of the potential risk of infusion-related reactions, which can include hypertension, chills, shivering, and chest, abdominal, and back pain [see Warnings and Precautions (5.3)].

All publications, patents, and patent applications mentioned in this specification are herein incorporated by reference to the same extent as if each individual publication, patent, or patent application was specifically and individually indicated to be incorporated by reference.

1. An approved product comprising a pharmaceutical composition comprising 180 mg/mL of an FcRn antagonist, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, and 20.5 mg/mL sucrose, and water for injection, USP, at a pH of 6.0, wherein the FcRn antagonist is efgartigimod, or a biosimilar version thereof.

2. A single-dose vial comprising the approved product of claim 1.

3. A method of reducing serum IgG levels in a subject in need thereof, the method comprising administering to the subject an effective amount of the approved product of claim 1.

4. A method of treating gMG in a subject in need thereof, the method comprising administering to the subject an effective amount of the approved product of claim 1.

5. The method according to claim 4, wherein the approved product is administered to the subject using a phased dosing schedule comprising a first treatment cycle and one or more subsequent treatment cycles, wherein the first treatment cycle comprises weekly administration of the approved product for 4 weeks, wherein the one or more subsequent treatment cycles each comprise weekly administration of the approved product for 4 weeks, and wherein each of the one or more subsequent treatment cycles is administered to the subject ≥50 days from the start of the previous treatment cycle.

6. A method of treating gMG in a subject in need thereof, the method comprising:
(a) administering an approved product comprising efgartigimod, or a biosimilar version thereof, to the subject at a dose of 1,008 mg via subcutaneous injection;
(b) monitoring the subject for a hypersensitivity reaction selected from the group consisting of anaphylaxis and hypotension leading to syncope; and
(c) initiating an appropriate measure to mitigate the hypersensitivity reaction when detected.

7. The method according to claim 6, wherein the approved product comprises 180 mg/ml efgartigimod, or a biosimilar version thereof, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, 20.5 mg sucrose, and water for injection, USP, at a pH of 6.0.

8. The method according to claim 6, wherein the approved product is administered to the subject via subcutaneous injection over 30 to 90 seconds.

9. The method according to claim 6, wherein the approved product is administered to the subject using a phased dosing schedule comprising a first treatment cycle and one or more subsequent treatment cycles, wherein the first treatment cycle comprises weekly administration of the approved product for 4 weeks, wherein the one or more subsequent treatment cycles each comprise weekly administration of the approved product for 4 weeks, and wherein each of the one or more subsequent treatment cycles is administered to the subject ≥50 days from the start of the previous treatment cycle.

10. The method according to claim 6, wherein the anaphylaxis and/or hypotension occurs within 1 hour of administration of the approved product to the subject.

11. The method according to claim 6, wherein the subject is monitored for at least 30 minutes after administration of the approved product to the subject.

12. The method according to claim 6, wherein the appropriate measure comprises permanent discontinuation of treatment with the approved product and/or instituting appropriate measures or seeking medical attention.

13. A method of treating gMG in a subject in need thereof, the method comprising:
(a) administering an approved product comprising efgartigimod, or a biosimilar version thereof, to the subject at a dose of 1,008 mg via subcutaneous injection;
(b) monitoring the subject for an infusion-related reaction; and
(c) initiating an appropriate measure to mitigate the infusion-related reaction when detected.

14. The method according to claim 13, further comprising:
(d) rechallenging the subject with the approved product when the infusion-related reaction is mild-to-moderate, wherein the rechallenging comprises one or more of the following: close clinical observation, slower infusion rate, and pre-medication.

15. The method according to claim 14, wherein the rechallenging comprises close clinical observation, slower infusion rate, and pre-medication.

16. The method according to claim 13, further comprising initiating appropriate therapy when the infusion-related reaction is severe.

17. The method according to claim 13, wherein the infusion-related reaction comprises one or more of the following: hypertension, chills, shivering, thoracic pain, abdominal pain, and back pain.

18. The method according to claim 13, wherein the approved product comprises 180 mg/mL efgartigimod, or a biosimilar version thereof, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, 20.5 mg sucrose, and water for injection, USP, at a pH of 6.0.

19. The method according to claim 13, wherein the approved product is administered to the subject via subcutaneous injection over 30 to 90 seconds.

20. The method according to claim 13, wherein the approved product is administered to the subject using a phased dosing schedule comprising a first treatment cycle and one or more subsequent treatment cycles, wherein the first treatment cycle comprises weekly administration of the approved product for 4 weeks, wherein the one or more subsequent treatment cycles each comprise weekly administration of the approved product for 4 weeks, and wherein each of the one or more subsequent treatment cycles is administered to the subject ≥50 days from the start of the previous treatment cycle.

21. The method according to claim 13, wherein the infusion-related reaction occurs within 1 hour of administration of the approved product to the subject.

22. The method according to claim 13, wherein the subject is monitored for at least 30 minutes after administration of the approved product to the subject.

23. An approved product comprising efgartigimod and hyaluronidase, wherein the product is approved for treatment of gMG in adult patients who are anti-AChR antibody positive.

24. The approved product of claim 23, wherein the product is provided in a single-dose vial containing 180 mg/mL efgartigimod, 2,000 U/mL rHuPH20, 1.4 mg/mL L-histidine, 2.2 mg/mL L-histidine hydrochloride monohydrate, 1.5 mg/mL L-methionine, 0.4 mg/mL polysorbate 20, 5.8 mg/mL sodium chloride, 20.5 mg sucrose, and water for injection, USP, at a pH of 6.0.

25. The approved product of claim 23, wherein the product induces an MG-ADL response rate of 67.7% and a QMG response rate of 63.1% in a population of gMG patients who received 10 mg/kg efgartigimod, compared to a MG-ADL response rate of 29.7% and a QMG response rate of 14.1% in a population of gMG patients who received placebo.

26. A biosimilar of the approved product of claim 23.

27. A biological product that is bioequivalent to the approved product of claim 23.

28. A kit comprising:
(a) the approved product of claim 23, and
(b) a label.

29. The kit of claim 28, wherein the label:
(a) includes a contraindication in patients with serious hypersensitivity to efgartigimod alfa products, to hyaluronidase, or to any excipients in formulations thereof;
(b) includes a warning for hypersensitivity reactions selected from anaphylaxis and hypotension leading to syncope;
(c) includes a warning for infusion-related reactions selected from hypertension, chills, shivering, thoracic pain, abdominal pain, and back pain;
(d) states to initiate appropriate therapy when a severe infusion-related reaction occurs;
(e) states that patients may be rechallenged with close clinical observation, slower infusion rates, and pre-medications when a mild-to-moderate infusion-related reaction occurs; and/or
(f) includes data demonstrating an MG-ADL response rate of 67.7% and a QMG response rate of 63.1% in a population of gMG patients who received 10 mg/kg efgartigimod, compared to a MG-ADL response rate of 29.7% and a QMG response rate of 14.1% in a population of gMG patients who received placebo.

30. A method of treating gMG in a subject in need thereof, the method comprising administering the approved product of claim 23 to the subject.


SEQUENCE LISTING (25 sequences)

SEQ ID NO: 1 - protein, 221 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 2 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 4 - protein, 222 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 5 - protein, 225 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 6 - protein, 226 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 7 - protein, 225 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 8 - protein, 224 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 9 - protein, 225 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 10 - protein, 224 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 11 - protein, 223 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 12 - protein, 223 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 13 - protein, 222 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 14 - protein, 221 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 15 - protein, 221 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 16 - protein, 220 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 17 - protein, 219 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 18 - protein, 224 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 19 - protein, 223 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 20 - protein, 222 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 21 - protein, 220 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 22 - protein, 227 aa, synthetic construct
  VARIANT 1: note = D or absent
  VARIANT 2: note = K or absent
  VARIANT 3: note = T or absent
  VARIANT 4: note = H or absent
  VARIANT 5: note = T or absent
  VARIANT 6: note = C or absent
  VARIANT 226: note = G or absent
  VARIANT 227: note = K or absent
XXXXXXPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPXX

SEQ ID NO: 23 - protein, 15 aa, synthetic construct
EPKSCDKTHTCPPCP

SEQ ID NO: 24 - protein, 509 aa, synthetic construct
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 25 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCPNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY